(19) United States
(12) Patent Application Publication
(10) Pub. No.: US 2011/0257289 A1
(43) Pub. Date: Oct. 20, 2011

(54) AB DIBLOCK COPOLYMERS AND APPLICATIONS FOR THEIR USE

(75) Inventors: Simon Biggs, Wetherby (GB); Gaelle Baquey, Verberie (FR); Maggy Manguian, Livry-Gargan (FR); Sasha Heriot, Doncaster (GB)

(73) Assignee: Chamelic Ltd, Leeds (GB)

(21) Appl. No.: 13/078,576

(22) Filed: Apr. 1, 2011

Related U.S. Application Data
(63) Continuation of application No. PCT/GB2009/002374, filed on Oct. 5, 2009.

(30) Foreign Application Priority Data
Oct. 3, 2008 (GB) 0818051.5
Oct. 24, 2008 (GB) 0819484.7

(51) Int. Cl. C09D 143/02; B05D 1/02; B05D 1/28; B05D 1/30; C09D 133/10; B05D 1/36; C09D 133/16; B05D 1/18; B05D 3/12; B82Y 5/00

(52) U.S. Cl. 523/102; 427/430.1; 427/427.4; 427/429; 427/240; 427/420; 427/435; 427/440; 427/439; 427/443.2; 427/427.5; 524/556; 427/407.1; 524/555; 524/547; 977/700

(57) ABSTRACT

The present invention relates to the use of AB block copolymer composition as a surface coating wherein the composition comprises (a) an AB block copolymer; and (b) a liquid medium and wherein the AB block copolymer comprises: (a) a substantially hydrophobic block A, and (b) a substantially hydrophilic block B wherein the hydrophobic block A comprises one or more monomer of formula (A) wherein R is H or C₁ to C₄ alkyl; Z is O, P or N; and R′ is selected from the group comprising: C₁ to C₁₈ linear or non linear alkyl; C₁ to C₁₈ alkylamino alkyl; C₁ to C₁₈ dihydroxyalkyl; C₁ to C₁₈ silylalkyl; epoxy alkyl, phosphoryl or phosphoryl alkyl; a styrene based monomer; a vinyl phosphonate or phosphoric acid monomer; and wherein the liquid medium comprises either: (i) water; (ii) an organic solvent; (iii) an organic solvent substantially free from water; or (iv) an organic solvent and water; and wherein: the liquid medium further optionally comprises one or more additive, surfactant or wetting agent.

### RELATED APPLICATIONS

This application is a continuation of PCT/GB2009/002374 filed on Oct. 5, 2009, which claims priority to GB 0819484.7, filed Oct. 24, 2008 and GB 9818051.5, filed Oct. 3, 2008, all of which is hereby incorporated by reference in their entirety.

### SUMMARY OF THE INVENTION

The present invention relates to the use of AB diblock copolymers and composition thereof as surface treatments. More specifically, the present invention relates to the novel use of AB diblock copolymers and compositions thereof which self assemble into aggregate structures in a suitable medium, and to a method suitable for preparing a surface treatment using same which provides functional benefits associated with easy-clean surface treatments such as dirt-repellency, spot-free finishes and anti-fogging properties. In addition, the treatment of surfaces with the AB diblock copolymers according to the present invention supplementary benefits such as anti-bacterial or anti-fungal properties.

### BACKGROUND TO THE INVENTION

The controlled wetting of surfaces has many potential applications such as the waterproofing of clothes and fabrics, concrete or paints, windows and windshields. In addition, controlled solid-liquid interfacial properties can have benefits in producing low friction surfaces for use in areas such as swimsuits, diving gear, boats and ships, as well as micro-fluidic devices.

Further applications in the area of “easy-to-clean” surfaces/coatings are also possible (1, 2). Such surfaces are usually designed to facilitate cleaning by minimising the adhesion of dirt and promoting water repellence such that as water “rolls off” the surface it collects the poorly adhered dirt particles (3). Often, “self-cleaning surfaces” such as those described in WO 96/04123 are termed “Lotus-effect” surfaces or coatings, and the technology is termed “Lotus-Effect” technology. Such “self-cleaning surfaces” can be produced by different ways: by creating the surface structures directly from hydrophobic polymers during manufacture or by creating the surface structures after manufacture (specifically by imprinting or etching, or by the adhesion of a polymer made of hydrophobic polymers to the surfaces).

In the present invention, the terms self-cleaning, easy-to-clean and stay-clean have a specific meaning.

The term self-cleaning surface treatment or coating, can be used to describe two possible situations.

The first definition relates to a self-cleaning surface treatment or coating which promotes the removal of dust and or dirt on a surface by means of water droplets rolling off the treated surface.

The second definition relates to a self-cleaning surface treatment or coating which is able to degrade dust and or dirt present on a surface and the residues thus produced are removed by a rinsing with water.

In both of the cases above a self-cleaning treatment or coating does not refer to a surface which repels dust/dirt; both of the situations above require water to remove dust and/or dirt from the surface.

The terms ‘stay-clean’ or ‘easy-clean’ surface treatment or coating both refer to a surface which repels dust and/or dirt. That is, prevents the build up of dust and/or dirt on a treated surface. The term ‘stay-clean’, is also used to refer to a treated surface which remains shiny after contact with water. Therefore, ‘stay-clean’ treatments prevent the formation of water streaks and/or water spots. In addition, a ‘stay-clean’ treatment or coating does not necessary need water for the surface to remain clean and shiny.

A variety of methods for controlling the wetting of surfaces have been reported, (4,5) based on both the control of the surface chemistry and the surface morphology (6). More recently, combinations of these two approaches have been used (3, 7). It is known for example from basic surface wetting theory that a low energy surface (with a concomitant large contact angle, greater than 100°) will tend to repel water. The result will be the formation of drops that roll off the surface easily.

U.S. patent application publication number 2002/0048679 (and related European patent application number EP 1018531) describe surfaces from which water runs off easily as having to be either very hydrophilic or hydrophobic. Hydrophilic surfaces have low contact angles with water, and this brings about rapid distribution of the water on the surface and finally rapid run-off of the resultant film of water from the surface. In contrast, hydrophobic surfaces form droplets through large contact angles with water. These droplets can roll off rapidly from inclined surfaces.

Many materials are known to be capable of producing water repellence. In general the materials possess a very low dielectric constant and are uncharged organics. Amongst these are materials such as halogenated organic polymers, for example polytetrafluoroethylene (PTFE) and derivatives thereof (8). One approach for manufacturing such surfaces is to apply a thin layer of a new material with the appropriate characteristics, for example appearance, durability, adhesion, and application requirements, directly onto the surface of interest. Such surface coatings or surface treatments should be easily and uniformly applied; established within a reasonable amount of time and process constraints; have a minimal environmental impact with respect to their synthesis and application; resist the effects of environmental assault; and provide good economic value.

The main problems with such materials to date include;

(a) Determining the best method to deposit the materials onto a surface of interest since the materials are often soluble in a limited number of organic solvents. One possibility is for example a spin-casting method. However, this method usually requires the liberal use of solvents with the associated cost and environmental concerns.

(b) The durability of the coatings when applied and used in ‘real applications’ is an issue. Damage of the coatings through abrasion and the impact of harsh external conditions can compromise their efficiency. For example, re-coating can not only be difficult but also expensive and is still subject to the same environmental concerns.

(c) Photodegradation effects caused by sunlight can also compromise the surface integrity and lead to re-application needs.

Random fluorinated copolymers prepared by radical copolymerisation of monomers in solution in a water-miscible organic solvent using peroxides or azo compounds as initiators have been described (see, for example, EP 542598, U.S. Pat. No. 1,106,630 and US 2004026053), together with their hydrophobic and oleophobic properties on various substrates.

U.S. Pat. No. 5,324,566 describes the use of hydrophobic fluorinated siloxane polymers for producing water repellent surfaces. It is disclosed in this patent that the water repelling properties of the fluorinated siloxane material can be improved by forming surface irregularities on the surface of such a material. It is for example mentioned that the surface is modified with irregularities of a height from about 0.1 micrometers up to the wavelength of visible light. Likewise, U.S. Pat. No. 5,599,489 and EP 0933388 A2 describe how the structured surface includes fluorine containing polymers or has been treated using alkylfluorosilanes.

US 2002/0048679 describes surfaces having a smooth, extremely hydrophobic polymer film (for example, polytetrafluoroethylene) and surfaces having a smooth extremely hydrophilic polymer film as examples where water and dirt run off without forming droplets. US 2002/0048679 further describes how a ‘long-term’ hydrophobic coating may be formed by applying certain silane derivatives underneath a hydrophobic coating on a surface. Other self-cleaning surfaces are described in US 2002/0150723, US 2002/0150724, US 2002/0150725, US 2002/0150726, US 2003/0013795 and US 2003/0147932.

U.S. Pat. No. 3,354,022 discloses water repellent surfaces having a rough micro structure with elevations and depressions and a hydrophobic material based on a fluorine containing polymer. According to one embodiment, a surface with a self-cleaning effect can be applied to ceramic, brick or glass by coating the substrate with a suspension comprising of glass beads (diameter of 3 to 12 micrometres) and a fluorocarbon wax which is a fluoroalkyl ethoxymethacrylate polymer. Unfortunately, such coatings have a disadvantage in that they posses a low abrasion resistance and only a moderate self-cleaning effect.

Further developments of surface coatings that are designed to produce strongly hydrophobic surfaces include the use of copolymers, polymer blends and mixtures of polymers and nanoparticles (such as titanium dioxide, as described in U.S. Pat. No. 6,800,354, U.S. Pat. No. 7,112,621 B2, U.S. Pat. No. 7,196,043 and DE 10016485.4). For example, coated surfaces have been produced using fluorocarbon polymers that can give contact angles of up to 120°. Titanium dioxide (TiO₂) has also been used with such fluorinated surfaces. It is known that under UV irradiation the TiO₂ is photocatalytically active and can produce super-wetting properties as a result of water hydrolysis effects (9). However the addition of TiO₂ present with (fluoroalkyl)silane does not affect the hydrophobicity of the overall material; that is, the modified (fluoroalkyl)silane remains hydrophobic. Consequently, whilst a self-cleaning material has been generated, due to the hydrophobicity of the system, water is able to rinse away any dirt from a treated surface, but, will leave water marks on the surface of the material, thereby failing to produce a system which is both clean and free from water marks.

The preparation of such surfaces using nanoparticles suffers from several drawbacks including the use of organic solvents (U.S. Pat. No. 3,354,022) and the use of a subsequent heat treatment (U.S. Pat. No. 6,800,354). Thus, there is a need for a simple process including an aqueous system for producing surfaces that are “easy-to-clean” with water and are optically transparent (such surfaces are not necessary hydrophobic, but can also be hydrophilic as illustrated in US 2002/0048679).

It has also been demonstrated recently that the control over surface wetting can be improved by producing surfaces with a well-controlled micron-sized roughness (10). These rough surface features assist in producing ‘ultrahydrophobic’ substrates by physical methods that include trapping air and reducing contact areas between the water drops and the surface. The basic underlying surface should itself be hydrophobic and when combined with the roughness effects, it results in surfaces with contact angles greater than 150° which are extremely hydrophobic. However, such surfaces are difficult to manufacture, they usually are very fragile and easily damaged and the micron-scale features can cause diffraction effects with light, which can be therefore problematic for use in applications involving glass.

Despite the above difficulties, products based upon such technology (or derivatives thereof) are beginning to appear in the market place. This is especially true for “self-cleaning” glass surfaces where random roughness is used to overcome diffraction problems. It is still unclear however, how effective these surfaces will be over time.

In addition to possessing an “easy-to-clean” surface, it is often desirable for the surface to possess an aesthetically pleasing finish, for example spot-free, streak-free or shiny finish that lasts for a reasonable period of time (for example from weeks to months). There are a number of waxes and other products currently in the market which attempt to give a spot-free finish. Typically, these products are designed to hydrophobically modify surfaces so that rainwater and tap water will bead-up' on the treated surface. Nonetheless, it is quite obvious that the beading of water on such surfaces may actually increase the formation of water spots since the beads of water will leave deposits on the surface when they dry. Furthermore, products available on the market often require rinsing with water after use. Typically when the water dries on the surface watermarks, smears, streaks or spots are left behind. These water-marks may be due to the evaporation of water from the surface leaving behind deposits of minerals which were present as dissolved solids in the water (for example calcium, magnesium, sodium ions and salts thereof) or maybe due to deposits of water carried soils, or even remnants from cleaning products (for example soap scum). This problem is often further exacerbated by some cleaning compositions which modify the surface during the cleaning process in such a way that after rinsing, water forms discrete droplets or beads on the surface instead of draining away. These droplets or beads dry to leave noticeable spots or marks referred to as watermarks. A known way of solving this problem is to remove the water drops from the surface using a cloth or chamois before the watermarks form. However this drying process (that is wiping washed and rinsed surfaces) is time consuming and requires considerable physical effort in the overall washing/cleaning process. Furthermore, access to the surface may be a problem so that wiping the surface is not a viable option.

U.S. Pat. No. 5,759,980 (Blue Coral) describes a composition to eliminate the problem of watermarks on a car. The cleaning composition described therein comprises a surfactant package comprising a fluorosurfactant or a silicone surfactant and mixtures thereof; and a substantive polymer which is capable of bonding to a surface to make it hydrophilic.

DE-A-2161591 also describes a composition for cleaning cars wherein the surface is made hydrophilic by the application of amino-group containing copolymers such as polymeric ethyleneimines, polymeric dimethylaminoethylacrylate or methacrylate or mixed polymerisates.

It is believed however, that the polymers described in these documents may be removed from the surface during rinsing of the product from the surface. Hence the surface hydrophilicity that is allegedly provided by the composition described may also be removed from the surface after the first rinse.

U.S. Pat. No. 6,846,512 B2 (Procter and Gamble) also describes a system and method for cleaning and/or treating a surface and in particular the exterior surface of a vehicle, however the method requires the application of a non-photoactive nanoparticle coating composition to a surface. Such non-photoactive nanoparticles can be inorganic nanoparticles (oxides, silicates, carbonates, hydroxides, layered clay minerals and inorganic metal oxides).

Therefore, there is a need for a treatment which provides a surface with “easy-to-clean” properties by ensuring that in addition to the repellence of dust and dirt, water-spotting and or streaking is also prevented, even if the surface once rinsed is not physically wiped to remove residual water.

Whilst many commercial surface coatings based on solutions of polymers in organic solvents are produced by drop-casting or spin coating, alternatives that are based on chemical grafting of polymer films have recently been discussed. Using this approach, coatings that comprise of dense brush-like films of polymers which are chemically attached to a surface are produced. The polymers detailed herein can have controlled chemistry that produces the desired wettability characteristics. Furthermore, the inherent chemical variety available to the synthetic polymer chemist means that such layers can be produced with a wide variety of physical properties, as well as the opportunity for including a stimulus responsive surface.

Stimuli-responsive polymers (11) are polymers that are able to respond to small changes in their environment with a corresponding large change in a specific physical property. Typical stimuli include: temperature, pH, ionic strength, light-, electric- and magnetic fields. Some polymers respond to a combination of two or more of these stimuli. For coatings or surface treatments, stimulus responsive polymers have the potential to be used in a wide variety of applications where controlled changes in properties such as adhesion, lubrication, and wetting are required.

GB patent application number PCT/GB2007/004762 describes novel AB block copolymers comprising of a fluorinated and a non-fluorinated portion. However, the use of fluorinated compounds can lead to difficulties in preparing water-based treatments. In addition, there are issues around the environmental impact of using such compounds and their formulation, as detailed below.

First of all, the use of fluorinated compounds has associated environmental concerns for applications in some commercial products. Public concern over the use of fluorinated compounds is high, and this can have a detrimental impact on the appeal of products that use such materials. Hence, there is a need for an alternative surface treatment composition that is more environmentally friendly to use.

Secondly, in general, environmental concern means that it is desirable to have a water-based formulation or a formulation having a very low volatile organic component. As it may sometimes be difficult to use fluorinated materials in aqueous formulations due to the low solubility of such compounds in water; using non-fluorinated materials provides more formulation options.

In addition, and importantly, the wide range of non-fluorinated monomers available means it is possible to fine-tune the “solubility” effectively through the use of different monomers. In comparison, the number of “fluorinated polymers” available are limited to only a few commercially available monomers. Hence, the use of non-fluorinated copolymers can provide improved formulations.

Easy-clean surface treatments need to be applied on various types of substrates. Therefore the compatibility of the materials with different substrates is also a key parameter. Non-fluorinated and especially alkyl materials provide a wider range of surface energies and therefore an ability to ‘tune’ the longevity of the effect, which is desirable for different applications. For example, building product applications may require up to ten years longevity whereas in homecare applications one to four weeks may be desirable. Thus, there is a need to have improved formulations that provide a better ability to tune these features. This is not available through the fluorinated materials described in the prior art.

It is therefore an object of the present invention to provide novel compositions that can be deposited onto a substrate surface with or without the need for organic solvents and which overcome the drawbacks of existing compositions in terms of cost of raw materials, production cost, environmental concerns and formulation in water based system.

It is a further aim of the present invention to provide a novel surface treatment that promotes variable wetting properties on the surface, or in other words provides an “easy-to-clean” surface meaning that an identifiable cleaning benefit (“easier-to-clean”, “cleaner-longer”, “stay-clean” etc.) on a surface will be observed by the end-user.

It is still a further aim of the present invention to provide novel compositions that are able to demonstrate a variable longevity of effect which is desirable for different application areas.

It is still a further aim of the present invention to provide novel compositions that are able to demonstrate a durable hydrophilic effect showing improved and homogenous water-sheeting and which are also able to provide a rapid drying surface which dries uniformly.

The surface treatment of the present invention is both an easy-clean treatment and a stay-clean treatment based on the definitions previously defined with significant advantages when compared to alternative hydrophobic surfaces and to self-cleaning windows, the latter not working for example in dry conditions. In the present invention, the excellent wetting results in a very thin and continuous water film when the surface is wet. When the liquid dries, any minerals in the water (for example lime) are spread uniformly in an extremely thin layer on the surface, which gives a spot free finish appearance. In addition, because the layer of water is so thin, it promotes a fast drying time aiding the formation of a spot-free finish

Therefore according to a first aspect of the present invention there is provided the use of an AB block copolymer composition as a surface coating wherein the composition comprises (a) an AB block copolymer; and (b) a liquid medium and wherein the AB block copolymer comprises:
(a) a substantially hydrophobic block A, and
(b) a substantially hydrophilic block B wherein
the hydrophobic block A comprises one or more monomer of formula A

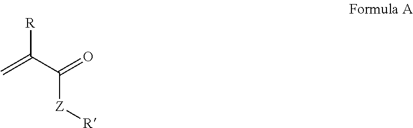

wherein R is H or C₁ to C₄ alkyl; Z is O, P or N; and
R′ is selected from the group comprising: C₁ to C₁₈ linear or non linear alkyl;
C₁ to C₁₈ alkylamino alkyl; C₁ to C₁₈ alkoxyalkyl; C₁ to C₁₈ dihydroxyalkyl; C₁ to C₁₈ silylalkyl; epoxy alkyl, phosphoryl or phosphoryl alkyl; a styrene based monomer; a vinyl phosphonate or phosphoric acid monomer; and wherein the liquid medium comprises either:
(i) water;
(ii) an organic solvent;
(iii) an organic solvent substantially free from water; or
(iv) an organic solvent and water; and wherein:
the liquid medium further optionally comprises one or more additive, surfactant or wetting agent.

In the AB block copolymer composition used according to the present the hydrophilic block B comprises one or more monomer of Formula B

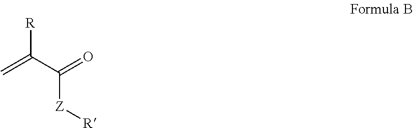

wherein R is H or C₁ to C₄ alkyl;

Z is O, N or P; and

R′ is selected from the group comprising: H; a C₁ to C₁₇ alkyl group with a pendent phosphoryl group, hydroxy group, silyl group, epoxy group or amine group.

When the hydrophobic block A comprises an alkylacrylic or acrylate monomer for Formula A, and R is H or C₁ to C₄ alkyl, and R′ comprises a C₁ to C₁₈ linear or non linear alkyl group of Formula 1

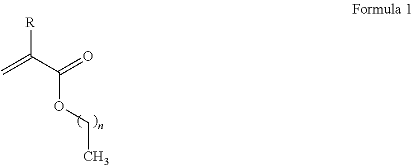

n is 1 to 11, more preferably 1 to 5.

When the hydrophobic block A comprises a monomer for Formula A, and R is H or C₁ to C₄ alkyl, and R′ comprises a C₁ to C₁₈ alkylamino alkyl group as shown in Formula 2

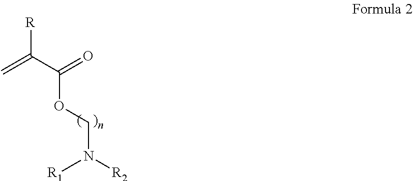

R₁ and R₂ are each independently: H; C₁ to C₆ alkyl group; phenyl; benzyl or cyclohexyl and n is 1 to 17, more preferably, R₁ and R₂ are each independently a C₁ alkyl group and n is 1 to 5.

When the hydrophobic block A comprises an alkylacrylic or acrylate monomer for Formula A, and R is H or C₁ to C₄ alkyl, and R′ comprises a C₁ to C₁₈ alkoxyalkyl group as in Formula 3a or 3b, n is 1 to 18, more preferably 1 to 4 and x and y are each independently 0 to 16, more preferably 0 to 6.

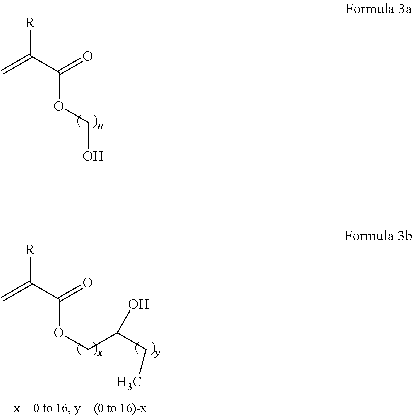

When the hydrophobic block A comprises an alkylacrylic or acrylate monomer for Formula A, and R is H or C₁ to C₄ alkyl, and R′ comprises a dihydroxyalkyl group as shown in Formula 4a or 4b, x and y is each independently 0 to 17 or 0 to 16 in Formula 4a and Formula 4b respectively, more preferably x and y is each independently 0 to 7 in Formula 4a or 0 to 6 in Formula 4b.

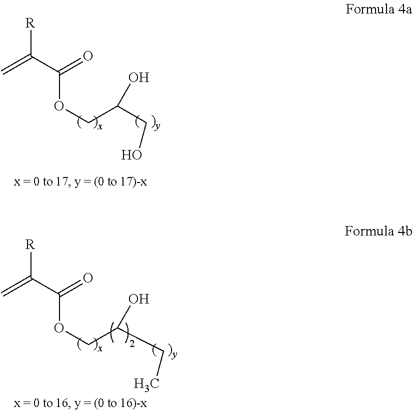

When the hydrophobic block A comprises an alkylacrylic or acrylate monomer for Formula A, and R is H or C₁ to C₄ alkyl, and R′ comprises a C₁ to C₁₇ silylalkyl group as shown in Formula 5a or 5b, R₁ is H or C₁ to C₄ alkyl and x and y are each independently 0 to 16, more preferably 1 to 6.

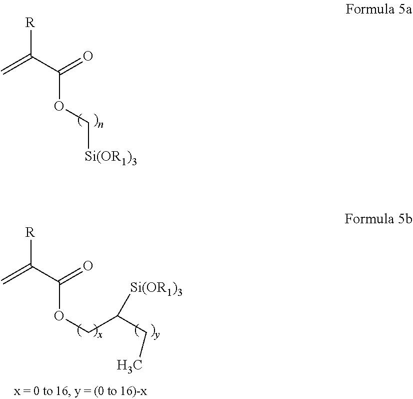

When the hydrophobic block A comprises an alkylacrylic or acrylate monomer for Formula A, and R is H or C₁ to C₄ alkyl, and R′ comprises an epoxy alkyl group as shown in Formula 6a or 6b, x and y are each independently 0 to 16, more preferably 0 to 6.

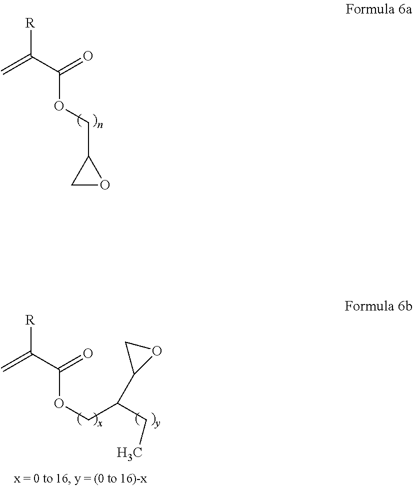

When the hydrophobic block A comprises a monomer of Formula A, and R is H or C₁ to C₄ alkyl group, and R′ comprises a phosphoryl or phosphoryl alkyl group as shown in Formula 7a and 7b, R₁ is H or C₁ to C₆ alkyl, more preferably H or C₁ alkyl.

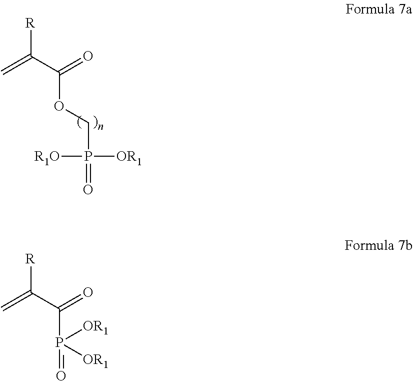

When the hydrophilic block B comprises an alkylacrylic acid or acrylic acid, R is H or C₁ to C₄ alkyl as shown in Formula 8

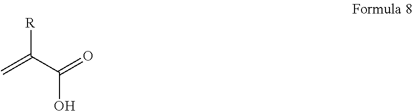

When the hydrophilic block B comprises a monomer of Formula B, R is H or C₁ to C₄ alkyl and R′ comprises C₁ to C₁₇ alkyl group with a pendent amine or amide group as shown in Formula 9a, 9b, 9c and 9d; n is 0 to 17, and R₁, R₂, R₃ and R₄ are each independently H; linear or non-linear C₁ to C₆ alkyl group, phenyl, benzyl or cyclohexyl, most preferably R₁, R₂, R₃ and R₄ are each independently C₁ to C₄ alkyl and X⁻ is chosen from the group selected from Cl, Br, I, ½SO₄, HSO₄ and CH₃SO₃, sulfonate, sulphate, carboxylate (acetate, glycolate), hydroxide, or phosphate.

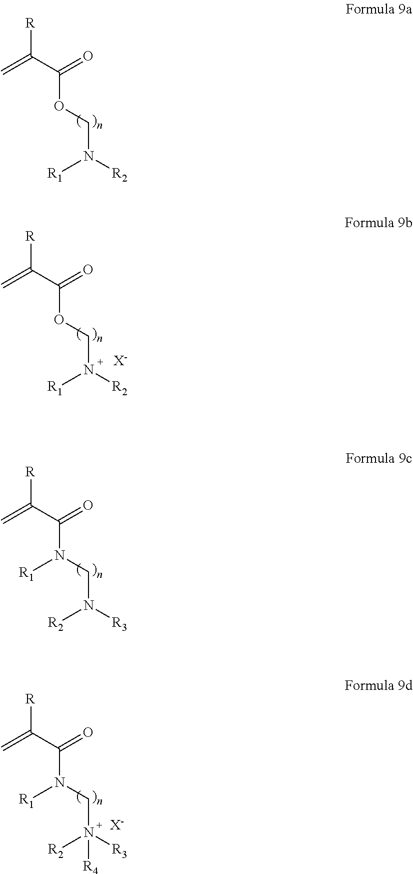

When the hydrophilic block B comprises a monomer of Formula B, R is H or C₁ to C₄ alkyl and R′ comprises C₁ to C₁₇ alkyl group with a pendent phosphoryl or phosphoryl alkyl as shown in Formula 10a or 10b, R₁ is H or C₁ to C₆ alkyl, most preferably H or C₁ alkyl.

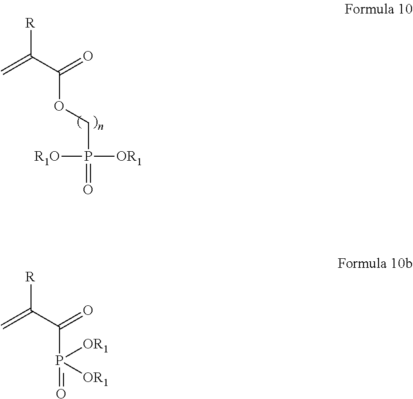

When the hydrophilic block B comprises a monomer of Formula B, R is H or C₁ to C₄ alkyl and R′ comprises C₁ to C₁₇ alkyl group with a pendent hydroxyl group as shown in Formula 11a, 11b, 11c and 11d n is 1 to 16.

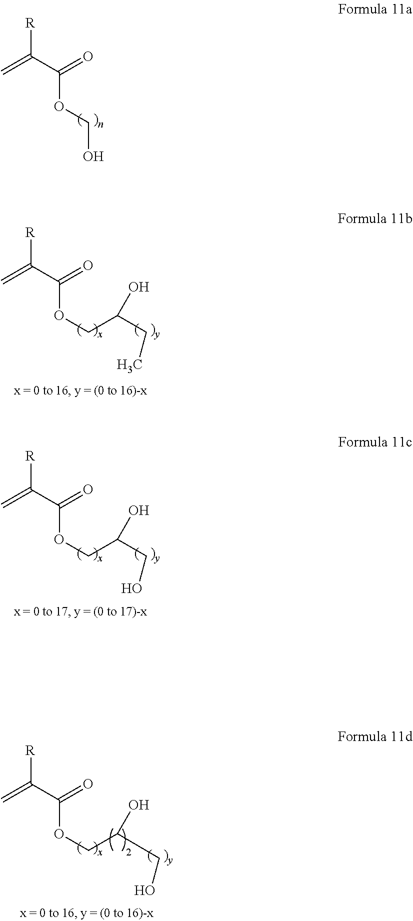

When the hydrophilic block B comprises a monomer of Formula B, R is H or C₁ to C₄ alkyl and R′ comprises C₁ to C₁₇ alkyl group with a pendent silyl group as shown in Formula 12a, and 12b, x and y are each independently 0 to 16 and n is 1 to 6.

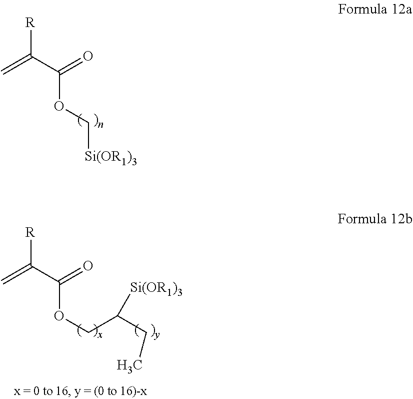

When the hydrophilic block B comprises an alkylacrylate or acrylate of Formula B, R is H or C₁ to C₄ alkyl and R′ comprises C₁ to C₁₇ alkyl group with a pendent epoxy group as shown in Formula 13a, and 13b, x and y are each independently 0 to 16 and n is 1 to 17.

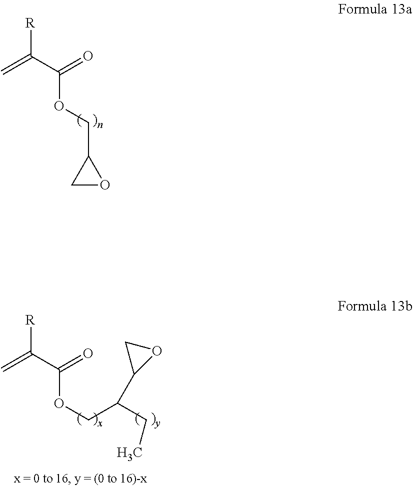

In accordance with the present invention the polymers comprising the AB block copolymer are comprised of monomers, and the ratio of the monomers comprising each polymer of the block copolymer AB is such that the volume fraction of the hydrophobic block A and the hydrophilic block B leads to the formation of organised aggregates. In addition, the volume fraction of the hydrophobic block A and the hydrophilic block B leads to the formation of micelles.

The ratio of the monomers comprising the block copolymer AB comprises: 5 to 100 units of A; and 15 to 300 units of B.

More preferably the ratio of the monomers comprising the block copolymer AB comprises: 15 to 50 units of A; and 80 to 200 units of B.

Even more preferably the ratio of the monomers comprising the block copolymer AB comprises: 15 to 30 units of A; and 100 to 120 units of B.

As stated above AB Block copolymers comprise a hydrophobic (“water hating”) block A and a second hydrophilic (“water loving”) block B. Variation in the copolymer properties can be obtained by varying the monomer types (different available chemistries), the molecular weights of the copolymer (at a fixed ratio of the two component block sizes), and the ratio of the molecular weights of the constituent blocks (at a fixed overall molecular weight for the copolymer).

Importantly, to form micelles (that is aggregates formed by molecules with an amphiphilic character) in aqueous media, the insoluble (or poorly soluble in water) hydrophobic blocks drive the formation of aggregates of the molecules. The structures of the aggregates are dependent on the copolymer concentration and the exact nature of the copolymer molecules. In the present invention copolymers are utilised that form spherical aggregates at the concentrations employed.

The coronal chemistry for the micellar aggregates should be such that the micelles will adsorb freely onto a wide variety of surfaces.

The substantially hydrophobic block A preferably comprises one or more monomers selected from the group comprising: alkyl alkylacrylate, alkylaminoalkyl alkylacrylate and a sillylacrylate. More preferably the substantially hydrophobic block A comprises an alkyl alkylacrylate selected from the group comprising a methacrylate; butyl methacrylate (BuMA) and octadecylmethacrylate (ODA). More preferably the hydrophobic block A comprises one or more alkylaminoalkylacrylate alkylacrylate monomer according to Formula A; wherein the alkylaminoalkylacrylate alkylacrylate monomer comprises an alkylaminoalkyl methacrylate or a diethylaminoethylmethacrylate (DEAEMA).

When block A comprises a silyl (alkyl)acrylate monomer, the silyl (alkyl)acrylate monomer preferably comprises: a trialkoxysilyl group, more preferably, a trimethoxysilyl group, most preferably (trimethoxysilyl) propyl methacrylate (TMSPMA) and (trimethoxysilyl) propyl acrylate (TMSPA).

When block A comprises a styrenic derivatives, the styrenic derivative preferably comprises styrene, methyl styrene, or styrenic derivatives substituted in ortho, meta, and/or para positions.

In addition, block A is preferably comprised of:
a homopolymer of an acrylate derivative;
a homopolymer of styrenic derivatives; and
a random, alternating, gradient or block copolymer based on A1A2, A1A3, A1A4, A2A4 or A3A4 wherein A1 is an alkyl acrylate; A2 an alkylamino acrylate; A3 a siliyl acrylate; and A4 a styrenic derivative.

A number of chemicals may be employed for the hydrophilic component B, all of which need to be water-soluble. Examples of such chemicals include, but are not limited to one or more polymers selected from the group comprising: hydrophilic organic monomers, oligomers, prepolymers or copolymers derived from vinyl alcohol, N-vinylpyrrolidone, N-vinyl lactam, acrylamide, amide, styrenesulfonic acid, combinations of vinylbutyral and N-vinylpyrrolidone, methacrylic acid, acrylic acid, vinylmethyl ether, vinylpyridylium halide, melamine, maleic anhydride/methyl vinyl ether, vinylpyridine, ethyleneoxide, ethyleneoxide ethylene imine, glycol, vinyl acetate, vinyl acetate/crotonic acid, methyl cellulose, ethyl cellulose, carboxymethyl cellulose, hydroxyethyl cellulose, hydroxypropyl cellulose, hydroxymethyl ethyl cellulose, hydroxypropylmethyl cellulose, cellulose acetate, cellulose nitrate, hydroxyalkyl(alkyl)acrylate such as hydroxyethyl(meth)acrylate, hydroxypropyl(meth)acrylate, alkylaminoalkyl(alkyl)acrylate, 2-(dimethyl amino)ethyl methacrylate, 2-(diethyl amino)ethyl methacrylate, 2-(diisopropyl amino)ethyl methacrylate, 2-(N-morpholino)ethyl methacrylate, or a derivative thereof, ethylene glycol(meth)acrylates (for example triethylene glycol(meth)acrylate) and (meth)acrylamide), N-alkyl(meth)acrylamides (for example N-methyl(meth)acrylamide and N-hexyl(meth)acrylamide), N,N-dialkyl(meth)acrylamides (for example N,N-dimethyl(meth)acrylamide and poly-N,N-dipropyl(meth)acrylamide), N-hydroxyalkyl(meth)acrylamide polymers, such as poly-N-methylol(meth)acrylamide and poly-N-hydroxy ethyl(meth)acrylamide, and N,N-dihydroxyalkyl(meth)acrylamide polymers, such as poly-N,N-dihydroxyethyl(meth)acrylamide, ether polyols, polyethylene oxide, polypropylene oxide, and poly(vinyl ether), alkylvinyl sulfones, alkylvinylsulfone-acrylates, (alkyl)acrylate with a pendent phosphorus group such as vinylphosphonate, vinylphosphonic acid, vinylphosphine oxide or any (alkyl)acrylate with a ester function —COOR such as R is CₓH₂ₓPO₃R₂ wherein x is 2 to 10, most preferably x is 2, and R is a hydrogen or an alkyl group having 1 to 4 carbon atoms, preferably methyl; and related compounds or a combination thereof.

In addition, the hydrophilic block B is comprised from monomers selected from the group consisting of: hydrophilic organic monomers, oligomers, prepolymers or copolymers derived from vinyl alcohol, N-vinylpyrrolidone, N-vinyl lactam, acrylamide, styrenesulfonic acid, combinations of vinylbutyral and N-vinylpyrrolidone, methacrylic acid, acrylic acid, vinylmethyl ether, vinylpyridylium halide, vinylpyridine, vinyl acetate, vinyl acetate/crotonic acid, hydroxyalkyl(alkyl)acrylate such as hydroxyethyl(meth)acrylate, hydroxypropyl(meth)acrylate, alkylaminoalkyl(alkyl)acrylate, 2-(dimethyl amino)ethyl methacrylate, 2-(diethyl amino)ethyl methacrylate, 2-(diisopropyl amino)ethyl methacrylate, 2-(N-morpholino)ethyl methacrylate, or a derivative thereof, ethylene glycol(meth)acrylates (for example triethylene glycol(meth)acrylate) and (meth)acrylamide), N-alkyl(meth)acrylamides (for example N-methyl(meth)acrylamide and N-hexyl(meth)acrylamide), N,N-dialkyl(meth)acrylamides (for example N,N-dimethyl(meth)acrylamide and poly-N,N-dipropyl(meth)acrylamide), N-hydroxyalkyl(meth)acrylamide polymers, such as poly-N-methylol(meth)acrylamide and poly-N-hydroxy ethyl(meth)acrylamide, and N,N-dihydroxyalkyl(meth)acrylamide polymers, such as poly-N,N-dihydroxyethyl(meth)acrylamide, ether polyols, polyethylene oxide, polypropylene oxide, and poly(vinyl ether), alkylvinyl sulfones, alkylvinylsulfone-acrylates, (alkyl)acrylate with a pendent phosphorus group such as vinylphosphonate, vinylphosphonic acid, vinylphosphine oxide or any (alkyl)acrylate with a ester function —COOR such as R is CₓH₂ₓPO₃R₂wherein x is 2 to 10, most preferably x is 2, and R is a hydrogen or an alkyl group having 1 to 4 carbon atoms, preferably methyl.

Furthermore, the hydrophilic block B is preferably comprised of monomers selected from the group consisting of: hydrophilic organic monomers, oligomers, prepolymers or copolymers derived from acrylamide, methacrylic acid, acrylic acid, hydroxyalkyl(alkyl)acrylate such as hydroxyethyl(meth)acrylate, hydroxypropyl(meth)acrylate, alkylaminoalkyl(alkyl)acrylate, 2-(dimethyl amino)ethyl methacrylate, 2-(diethyl amino)ethyl methacrylate, 2-(diisopropyl amino)ethyl methacrylate, 2-(N-morpholino)ethyl methacrylate, or a derivative thereof, N-alkyl(meth)acrylamides (for example N-methyl(meth)acrylamide and N-hexyl(meth)acrylamide), N,N-dialkyl(meth)acrylamides (for example N,N-dimethyl(meth)acrylamide and poly-N,N-dipropyl(meth)acrylamide), N-hydroxyalkyl(meth)acrylamide polymers, such as poly-N-methylol(meth)acrylamide and poly-N-hydroxy ethyl(meth)acrylamide, alkylvinyl sulfones, alkylvinylsulfone-acrylates and (alkyl)acrylate with a pendent phosphorus group such as vinylphosphonate, vinylphosphonic acid, vinylphosphine oxide or any (alkyl)acrylate with a ester function —COOR such as R is CₓH₂ₓPO₃R₂ wherein x is 2 to 10, most preferably x is 2, and R is a hydrogen or an alkyl group having 1 to 4 carbon atoms, preferably methyl.

More preferably the hydrophilic block B is comprised of monomers selected from the group of hydrophilic organic monomers, oligomers, prepolymers or copolymers derived from methacrylic acid, acrylic acid, alkylaminoalkyl(alkyl)acrylate, 2-(dimethyl amino)ethyl methacrylate, 2-(diethyl amino)ethyl methacrylate, 2-(diisopropyl amino)ethyl methacrylate, 2-(N-morpholino)ethyl methacrylate, or a derivative thereof.

The AB block copolymer may take the form of: linear block copolymer (diblock, triblock or multiblock), miktoarm copolymer (star copolymer), ladder (H-shaped) copolymer, graft and comb (co)polymer; preferably a linear block copolymer.

Also, the distribution of component monomers within each copolymer block is in the form of homo, random, gradient, alternative, block, graft and comb (co)polymers.

It is also preferred that the block copolymer is preferably selected from the group comprising: AB blocks, ABA blocks, ABC blocks copolymers.

In accordance with the present invention the block copolymer comprises at least one block that absorbs to a target surface. The composition may further comprise a primer. Also, the composition may form micelles and the aggregate structures of the composition have a diameter between 3 and 300 nm.

In a preferred example, the polymers used in the composition are prepared by controlled living radical polymerisation reactions.

Also in the composition used in accordance with the present invention the liquid medium may comprise water, water and organic solvent, an organic solvent, or an organic solvent free from water, and wherein the block copolymer is preferably completely dissolved in the liquid medium.

The organic solvent preferably comprises water-miscible organic solvents selected from the group comprising: C₁₋₆ alcohol, preferably, methanol, ethanol, n-propanol, iso-propanol, n-butanol, and sec-butanol; alkylketones, arylalkylketones, ketoalcohols, cyclic ketones, heterocyclic ketones, ethers, cyclic ethers, esters, and the like and combinations thereof.

The liquid medium preferably comprises: water or a mixture of water/alcohol or pure alcohol wherein the alcohol is preferably selected from the group comprising: methanol, ethanol, industrial methylated spirit, propanol, isopropanol, tertbutanol, ethylene glycol or glycol ethers.

The relative proportions of block copolymers AB as components (a) and of liquid medium as component (b) in the composition comprises between 1:100,000 to 1:1, more preferably from 1:10,000 to 1:2, and especially from 1:5,000 to 2:10. Most preferably the relative proportions of component (a) and component (b) in the composition comprises 1:5,000 to :1:10.

In addition, the composition may further comprise additional components selected from for example dispersants, perfumes surfactants and stabilisers.

The composition is preferably used to coat a substrate to by means of: dipping, spraying, wiping, spin coating, roller coating, curtain-flowing and brush coating.

The substrate may be selected from the group comprising: glass, plastics, metals, ceramics, concrete, paper, wood, minerals, painted and/or coated substrates or the substrate may be coated or painted prior to application of the composition with a primer.

The composition is preferably used to create a coating which has one or more of the following properties, water-sheeting, anti-fog, anti-dust, ant-bacterial and anti-fungal.

The polymers of the present invention may be in the form of, for example, homo, random, gradient, block (diblock, triblock or multiblock), copolymers, graft and comb (co)polymer. The component monomers within the copolymer may be dispersed randomly, alternately or in blocks. It is preferred however that the copolymer is a block copolymer.

The block copolymer is preferably selected from the group consisting: of AB blocks, ABA blocks, ABC blocks, comb, ladder, and star copolymers. However, it is most preferred that the block copolymer includes at least one block that is able to be adsorbed to the target surface

In some circumstances, it can be beneficial to use a pre-treatment such as a primer which can enhance the adhesion of the polymer to the surface. Indeed, a primer is a preparatory coating put on materials before painting or treating. Priming ensures better adhesion of paint or treatment/coating to the surface, increases paint or treatment/coating durability, and provides additional protection for the material being painted or treated/ coated. The primer allows finishing paint or treatment to adhere much better than if it was used alone. For this purpose, primer is designed to adhere to surfaces and to form a binding layer that is better prepared to receive the paint or treatment/coating. Hence, good adhesion of the polymer on a substrate can be achieved through the use of a primer and controlling the primer's physical properties such as porosity, tackiness, and hygroscopy.

It is also important that the structure of the material should lead to the formation of micelles (that are aggregates formed by molecules with an amphiphilic character; molecules having the tendency to aggregate into larger scale structures (3 to 300 nm) when certain conditions in their environment are changed, for example pH, salt concentration, temperature, solvent etc.) or micellar aggregates (that are micelles that have aggregate into a larger scale structure (3 to 300 nm) in aqueous media

Preferably, the block copolymers according to the first aspect of the present invention are prepared by means of controlled living radical polymerisation to obtain narrow molecular weight distribution copolymers. Suitable synthetic routes include but are not limited to: Reversible Addition-fragmentation chain transfer (RAFT), Group transfer polymerisation (GTP) and Atomic transfer radical polymerisation (ATRP), Activated regenerated by electron transfer (ARGET), nitroxide-mediated polymerization (NMP).

The block copolymers of the present invention may be available in solid or substantially solid form, for example a powder, or alternatively may be available as a liquid.

In the composition according to the present invention the liquid medium preferably comprises of a mixture of water and an organic solvent, or an organic solvent free from water and the block copolymer is preferably completely dissolved in the liquid medium

Organic solvents suitable for use in the composition of the present invention preferably comprise of water-miscible organic solvents selected from: C₁₋₆ alcohol, preferably, methanol, ethanol, n-propanol, iso-propanol, n-butanol, and sec-butanol; alkylketones, arylalkylketones, ketoalcohols, cyclic ketones, heterocyclic ketones, ethers, cyclic ethers, esters, and the like and combinations thereof.

Most preferably the solvent comprises water or a mixture of water/alcohol or pure alcohol where the alcohol is preferably chosen from the group comprising methanol, ethanol, industrial methylated spirit, propanol, isopropanol, tertbutanol, ethylene glycol or glycol ethers.

Organic solvents substantially free from water: the organic solvents for use in the composition of the present invention preferably include but are not limited to organic solvents selected from the group comprising: tetrahydrofuran, dichloromethane, ethyl acetate, chloroform, lower alcohols, ketones or dimethyl sulphoxide.

In addition, when the liquid medium comprises an organic solvent substantially free from water the composition preferably further comprises a suitable polar solvent.

Furthermore, it will be appreciated by one skilled in the art that in the composition of the present invention the organic solvent free from water may comprises a single organic solvent or a mixture of two or more organic solvents.

The relative proportions of block copolymers AB as components (a) and of liquid medium as component (b) in the composition of the present invention preferably comprises between 1:100,000 to 1:1, more preferably from 1:10,000 to 1:2, and especially from 1:5,000 to 2:10.

Most preferably the relative proportions of component (a) and component (b) in the composition comprises 1:5,000 to :1:10.

It will also be appreciated that the composition according to present invention may preferably further comprise additional components or auxiliary agents selected from for example but not limited to dispersants, perfumes, biocides, and stabilisers, surfactants or wetting agents, emulsifiers, colouring agents, dyes, pigments, UV absorbers, radical scavenger, antioxidant, anti-corrosion agent, optical brightener, fluorescers, bleaches, bleach activators, bleach catalysts, non-activated enzymes, enzyme stabilizing systems, chelants, coating aid, metal catalyst, metal oxide catalyst, organometallic catalyst, filmforming promoter, hardener, linking accelerator, flow agent, leveling agent, defoaming agent, lubricant, matte particle, rheological modifier, thickener, conductive or non-conductive metal oxide particle, magnetic particle, anti-static agent, pH control agents, perfumes, preservative, biocide, pesticide, anti-fouling agent, algicide, bactericide, germicides, disinfectant, fungicide, bio-effecting agent, vitamin, drug, therapeutic agent or a combination thereof.

The copolymers used according to the present invention have been found to form micelles in aqueous solutions. The coronal properties of those micelles can be tuned using triggers such as pH, temperature or salt concentrations; providing variable size of micelles and/or variable adhesion to the surface.

The compositions used in the present invention have many potential uses. The compositions are trigger-responsive (especially pH and salt concentration) and accordingly have potential for a wide variety of possible uses where controlled changes in surface properties, such as adhesion, lubrication and wetting are required.

The compositions used according to the present invention are particularly suitable for use as either a surface coating or a surface treatment to form an “easy-to-clean” surface. To this end, the present invention also provides a surface coating or a surface treatment prepared using the composition of block copolymer described according to the first aspect of the present invention.

There is also provided a method of coating a substrate comprising the steps of preparing a composition according to a first aspect of the present invention and exposing the substrate to the aqueous solution.

Preferably, the method includes gentle agitation of the copolymer molecules to fully dissolve the molecules in the composition. Preferably, the solution is left to equilibrate for up to 24 hours, prior to application.

Methods of exposing the substrate to the composition include any known technique for forming a coating from a solution, such as spin coating, dip coating, roller coating, brush coating, gravure coating, wiping, curtain flow or spraying.

Surfaces that can be treated with the composition of the present invention include, but are not limited to, glass, plastics, metals, ceramics, concrete, paper, wood, minerals, painted and/or coated substrates. Optionally, the substrate may be rinsed with a pure solvent, such as water, to remove any loosely held copolymer molecules.

The present invention will now be described and illustrated further by reference to the following examples and figures in which:

Example 1—describes a method for the preparation of block copolymers;

Example 2—investigates the coating of a substrate with the block copolymer of example 1;

Example 3—investigates the “easy-to-clean” properties and in particular the “dirt-repellency” properties of a substrate treated with the block copolymer of example 1;

Example 4—investigates the “water-sheeting” properties of a substrate treated with the block copolymer of example 1;

Example 5—investigates the “antifog” properties of a substrate treated with the block copolymer of example 1;

Example 6—describes the “Anti-spotting” properties of surface treatment according to the present invention

Example 7—investigates the “antibacterial and anti-fungal” properties of a substrate treated with the block copolymer of example 1;

Example 8—describes the improvement of a concentration of copolymers in water-based formulation using the non-fluorinated block copolymer of example 1 in comparison to a fluorinated copolymer;

Example 9—Comparison of a NF-AP (15/120)-based surface treatment compared to surface treatments prepared from existing additives which promotes stay-clean properties;

### BRIEF DESCRIPTION OF THE DRAWINGS

FIG. 1—illustrates the “easy-to-clean” properties of a substrate applied with a surface treatment according to the present invention.

FIG. 1a—illustrates the substrate after one dirt cycle.

FIG. 1b—illustrates the substrate after three dirt cycle.

FIG. 1c—illustrates the substrate after five dirt cycle as described in the present invention.

FIG. 2—is a graph showing colour difference ΔE versus the number of dirt cycles obtained by spectrophotometric measurements of the substrates from FIG. 1.

FIG. 3—is a photographic image illustrating the use of the surface treatment according to the present invention on a shower door in a bathroom.

FIGS. 4a, 4b and 4c—are photographic images illustrating the “water-sheeting” properties of a substrate applied with a surface treatment containing the polymer PBuMa₁₅-b-MAA₁₉ according to the present invention.

FIGS. 4a and 4b illustrate a PVC window frame panel and a black polyester powder coated aluminium panel half treated with a surface treatment from a pure alcoholic solution (ethanol) respectively.

FIG. 4c—illustrate a white polyester powder coated aluminium panel half treated with a water-based surface treatment.

FIGS. 5a and 5b—are photographic images illustrating the “water-sheeting” properties of a substrate applied with a surface treatment containing the polymer PBuMa₁₅-b-(TMACMA₆₀-co-DMAEMA₆₁) according to the present invention.

FIGS. 5a and 5b illustrate a PVC window frame panel and a black polyester powder coated aluminium panel half treated with a surface treatment from a pure alcoholic solution (ethanol) respectively.

FIGS. 6a and 6b—illustrates the “antifog” properties of a substrate applied with a surface treatment containing the polymer PBuMa₁₅-b-MAA₁₁₉ according to the present invention.

FIGS. 6a and 6b—illustrate a polyester film and a mirror half treated with a water-based surface treatment respectively.

FIG. 7—illustrates the “antifog” properties of a substrate applied with a surface treatment according to the present invention.

FIG. 8—illustrates the “anti-spotting” properties of a substrate applied with a surface treatment according to the present invention.

FIGS. 9, 10 and 11 illustrate the results of tests performed with polymers according to the present invention.

FIG. 12 illustrates the results of wetting tests performed with polymers according to the present invention in comparison to surface treatments prepared from commercially available existing additives.

### ABBREVIATIONS

| | |
|---|---|
| AIBN | 2,2′-azobis(2-methylpropionitrile) |
| nBuMA | n-butyl methacrylate |
| CPDB | 4-cyanopentanoic dithiobenzoate |
| DMAEMA | N,N′-dimethylaminoethyl methacrylate |
| MAA | Methacrylic acid |
| PBuMA | Poly(n-butylmethacrylate) |
| PMAA | Poly(methacrylic acid) |
| TMACMA | Methacryloyloxyethyl trimethyl ammonium chloride or |
| | N, N′, N″-trimethylammonium chloride methacrylate |

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

### EXAMPLE 1

### Preparation of Polymers and Block Copolymers According to the Present Invention

To manufacture the AB diblock copolymers of the present invention a controlled polymer or copolymer synthetic process is required in order to achieve a product with the required properties of, for example, desired molecular weight and narrow weight distribution or polydispersity. Polymers with a narrow molecular weight distribution are able to possess and exhibit substantially different properties to polymers prepared by conventional means.

Living radical polymerizations (also sometimes referred to as controlled free radical polymerizations) provide a maximum degree of control for the synthesis of polymers with predictable and well-defined structures. Recently, controlled/living radical polymerization (CLRP) has been shown to be a viable technique for the preparation of a large and diverse range of materials with precisely tailored nano- and micro-scale features.

Group transfer polymerisation (GTP), atom transfer radical polymerization (ATRP), nitroxide-mediated polymerization (NMP), reversible addition fragmentation transfer polymerization (RAFT), and MADIX all form part of these recently developed techniques and even more recently, Activated Regenerated by Electron Transfer (ARGET). These methods allow the synthesis of a large variety of polymeric architectures such as block copolymers, graft copolymers, stars, star-blocks, miktoarm stars or macromolecular brushes.

Importantly, a controlled polymerisation process is required for controlling the molecular structure of synthetic polymers, and thus for controlling the size of micellar aggregates in solution. Indeed, the aggregate sizes of polymers depend directly on the exact nature of the polymer (that is for example; molecular weight, length of polymer, and ratio between hydrophobic and hydrophilic blocks).

Whilst the presently described example prepares a block copolymer using the RAFT agent, CPDB (4-cyanopentanoic dithiobenzoate), it will be appreciated that other RAFT agents may be used. Similarly, the block copolymers of the present invention may be prepared by means of other controlled living polymerisation techniques as previously mentioned above.

For example when using Group transfer polymerisation (GTP), the polymerisation reaction as described in S. P. Rannard, N. C. Billingham, S. P. Armes and J. Mykytiuk, European Polymer Journal, 1993, vol 29 p 407. “Synthesis of monodisperse block copolymers containing methacrylic acid segments by group-transfer polymerization: choice of protecting group and catalyst”; may be followed, the relevant section of which is incorporated herein by reference.

For example when using atom transfer radical polymerization (ATRP), the polymerisation reaction as described in: Arrainen, Amilcar Pillay; Pascual, Sagrario; Haddleton, David M.; Journal of Polymer Science, Part A: Polymer Chemistry (2002), 40(4), 439-450. “Amphiphilic diblock, triblock, and star block copolymers by living radical polymerization: synthesis and aggregation behaviour” may be followed, the relevant section of which is incorporated herein by reference.

When using nitroxide-mediated polymerization (NMP), the polymerisation reaction as described in: WO 2007/057620 A1 (Arkema); “Method for preparing a living polymer comprising methacrylic and/or methacrylate units” may be followed, the relevant section of which is incorporated herein by reference.

However, it will be appreciated by one skilled in the art that modifications may be appropriate to the above methods to prepare a target polymer.

RAFT Synthesis of PBuMA₁₅-b-PMAA₁₁₉

In a first step, the following conditions were used for the synthesis of PBuMA with a targeted polymerization degree of 15. nBuMA (5.01 g, 35.24 mmol), 4-cyanopentanoic dithiobenzoate (CPDB) (0.518 g, 2.34 mmol), AIBN (0.19 g, 1.18 mmol) and propan-2-ol (solvent, 8.45 g, 60% in mass) were introduced in a 250 mL round bottom flask containing a magnetic stirrer. The reaction flask was degassed by nitrogen bubbling for 20 minutes at 0° C. and then heated at 75° C. in a thermostated oil bath under a nitrogen atmosphere. After 5 hours of polymerization, a sample was withdrawn to analyze by ¹H NMR and thus calculate the conversion of BuMA.

In a second step, a solution containing MAA (24.2 g, 0.28 mmol), AIBN (0.20 g, 1.25 mmol) and propan-2-ol (solvent, 70.2 g, 75% in mass) was prepared in a second round bottom flask. This solution was degassed by nitrogen bubbling for 20 minutes at 0° C. and was then transferred via a cannula to the reaction flask. After stirring for 17 hours at 70° C., the reaction was cooled via an ice bath in order to stop the polymerisation. A total conversion of MAA was determined by ¹H NMR spectroscopy in DMSO at 400 MHz, integrating one vinylic proton (at 5.9 to 6 ppm from MAA) in comparison with the peak at 12.3 ppm corresponding to COOH from both the monomer and the polymer). The polymer was then purified by precipitation in diethyl ether. The recovered polymer PBuMA₁₅-b-PMAA₁₁₉ was dried in a vacuum oven overnight at 40° C.

RAFT Synthesis of PBuMA₁₅-b-P(TMACMA₆₀-co-DMAEMA₆₁)

In a first step, PBuMA₁₅was prepared as previously described above from a mixture of nBuMA (2.56 g), CPDB (0.26 g), AIBN (0.09 g) and propan-2-ol (solvent, 4.52 g, 60% in mass). The polymerisation was allowed to proceed for 5 hours at 75° C. and a conversion of 95% obtained by ¹H NMR in CDCl₃.

In a second step, a degassed solution containing TMACMA (19.72 g), DMAEMA (11.43 g), AIBN (0.09 g) and propan-2-ol (35.23 g) was transferred via cannula into the reaction flask containing PBuMA₁₅. After stirring for 17 hours at 70° C., the reaction was cooled in an ice bath in order to stop the polymerisation. A total conversion of TMACMA and DMAEMA was determined by ¹H NMR spectroscopy in DMSO. The polymer was then purified by precipitation in cold hexane. The recovered polymer PBuMA₁₅-b-P(TMACMA₆₀-co-DMAEMA₆₁)was dried in a vacuum oven overnight at 40° C.

The same procedure as described above was followed for the synthesis of the copolymer PBuMA₁₅-b-PDMAEMA₁₂₀ using the appropriate starting materials.

RAFT Synthesis of P(BuMA₁₅-co-DEAEMA₁₅)-b-PDMAEMA₁₂₀)

In a first step, P(BuMA₁₅-co-DEAEMA₁₅) was prepared following the same procedure as for the preparation of PBuMA₁₅ above but using instead a mixture of nBuMA (2.53 g), N,N-diethylaminoethylmethacrylate (DEAEMA, 3.36 g), CPDB (0.26 g), AIBN (0.09 g) and propan-2-ol (solvent, 4.25 g, 60% in mass). The polymerisation was allowed to proceed for 5 hours at 75° C.

In a second step, a degassed solution containing DMAEMA (22.13 g), AIBN (0.09 g) and propan-2-ol (38.75 g) was transferred via cannula into the reaction flask containing P(BuMA₁₅-co-DEAEMA₁₅). After stirring for 24 hours at 70° C., the reaction was cooled in an ice bath in order to stop the polymerisation. The complete conversion of the reactants was determined by ¹H NMR spectroscopy in DMSO. The polymer was then purified by precipitation in cold hexane. The recovered polymer P(BuMA₁₅-co-DEAEMA₁₅)-b-PDMAEMA₁₂₀) was dried in a vacuum oven overnight at 40° C.

NMP Synthesis of PBuMA₁₅-b-PMAA₁₁₉

In a first step, the following conditions were used for the synthesis of PBuMA with a targeted polymerization degree of 15. nBuMA (15.01 g, 0.1 mol), styrene (1.12 g, 10.7 mmol), BlocBuilder® (2.68 g, 7.03 mmol), and propan-2-ol (solvent, 8.49 g, 70% in mass) were introduced in a 500 mL round bottom flask equipped with a mechanical stirrer. The reaction flask was degassed by nitrogen bubbling for 20 minutes at 0° C. and then heated at 75° C. in a thermostated oil bath under a nitrogen atmosphere. After 8 hours of polymerization, a sample was withdrawn to analyze by NMR and thus calculate the conversion of BuMA (conversion 73%).

At the end of this step, the reaction was left to cool down to 50° C. Then, propan-2-ol (solvent, 171.5 g) was added. The mixture was kept under stirring at room temperature overnight.

In a second step, MAA (73.4 g, 0.85 mol), styrene (7.41 g, 71.2 mmol) and AIBN (0.57 g, 3.48 mmol) were added to the previous mixture. The reaction mixture was degassed by nitrogen bubbling for 20 minutes at 0° C. After stirring for 4 hours at 75° C., a conversion of 92% in MAA was determined by ¹H NMR spectroscopy in DMSO at 400 MHz, integrating one vinylic proton (at 5.9 to 6 ppm from MAA) in comparison with the peak at 12.3 ppm corresponding to COOH from both the monomer and the polymer). IPA was evaporated and the recovered polymer PBuMA₁₅-b-PMAA₁₁₉ was dried in a vacuum oven overnight at 40° C.

NMP Synthesis of P(BuMA₁₅-co-DEAEMA₁₅)-b-PDMAEMA₁₂₀)

In a first step, PBuMA₁₅ was prepared following the same procedure as for the preparation of PBuMA₁₅ above but using instead a mixture of nBuMA (13.6 g), styrene (1.1 g), BlocBuilder® (2.42 g), and propan-2-ol (solvent, 5.8 g, 75% in mass). The polymerisation was allowed to proceed for 8 hours at 75° C.

In a second step, DMAEMA (59.7 g), TMACMA (105.2 g), styrene (7.3 g), AIBN (0.52 g) and propan-2-ol (solvent, 155.7 g) were added to the reaction flask. The mixture was degassed by nitrogen bubbling for 20 minutes at 0° C. After stirring for 4 hours at 75° C., the polymerization was stopped. IPA was evaporated and the recovered polymer PBuMA₁₅-b-PMAA₁₁₉ was dried in a vacuum oven overnight at 40° C.

The same procedure as described above was followed for the synthesis of the copolymer PBuMA₁₅-b-PDMAEMA₁₂₀ using the appropriate starting materials.

TABLE 1

| | Block A | | | Block B | | | | Preparation |
|---|---|---|---|---|---|---|---|---|
| Batch | BuMa | DEAEMA | Styrene | MAA | DMAEMA | TMACMA | Styrene | Method |
| 1 | 15 | 0 | 0 | 120 | 0 | 0 | 0 | RAFT |
| 2 | 30 | 0 | 0 | 120 | 0 | 0 | 0 | RAFT |
| 3 | 15 | 0 | 1.5 | 120 | 0 | 0 | 11 | NMP |
| 4 | 15 | 0 | 0 | 0 | 120 | 0 | 0 | RAFT |
| 5 | 15 | 15 | 0 | 0 | 120 | 0 | 0 | RAFT |
| 6 | 15 | 0 | 0 | 0 | 60 | 60 | 0 | RAFT |
| 7 | 15 | 0 | 1.5 | 0 | 60 | 60 | 11 | NMP |

### EXAMPLE 2

### Method Describing the Preparation and Application of a Surface Treatment According to the Present Invention

A polymer or polymeric composition prepared according to the present invention may be coated onto a preferred substrate as described hereafter by any established coating process, for example, but not limited to for example a spray process. Generally, the treatment process involves the following steps:

Step (1): Dissolution of the copolymer molecules in water or in a mixture of alcohol/water at a desired pH and salt conditions under gentle agitation. Typically the system is left to equilibrate for 24 hours.

The copolymers chosen are usually not of a high molecular weight (for example the copolymers typically have a range of between 2000 to 100000 g/mol) and such molecules equilibrate rapidly when dissolved in an aqueous solution or in a mixture of alcohol/water.

Solvents suitable for use in the composition of the present invention are preferably as previously described.

In the following experimental procedure, the copolymer systems were left for 24 hours simply to be certain that the systems were fully equilibrated. However, the system was also found to equilibrate within much shorter timescales. For example, in pure ethanol the copolymer system was ready to use after just two hours of stirring. In a mixture of water (92% w) and ethanol (8% w), it was necessary to first fully dissolve the polymer in ethanol for two hours and then to add the polymer to water and mix the system again for at least one further hour.

Agitation of the copolymer systems was required during the process of dissolution and mixing, but it was not found to be critical and simply slowly stirring the copolymer system was found to be sufficient. The length of time for agitation depended on the solvent system.

Step (2): Exposure of the substrate of interest to the copolymer solution, that is, applying the solution to a desired substrate.

Whilst not wishing to be bound by any particular theory, evidence from the present invention implies that adsorption is complete after a few minutes. Methods of exposing the substrate to the solution include for example any known technique for forming a coating from a solution such as spin coating, dip coating, roller coating, brush coating, curtain flow or spraying, roller coating, wire-bar coating, extrusion coating, air knife coating, curtain coating, slide coating. More preferably dipping and spraying ensures that every part of the surface has been wetted by the treatment composition.

The treatment can be applied both interior and exterior surfaces.

Step (3): Drying the treated surface.

Preferably the treated surfaces need to be dried after applying the treatment composition. This can be achieved at room temperature or at higher temperatures, but if higher temperatures are used the drying time should be reduced. It should be noted that the drying temperature does not enhance the performance of the coating; rather it shortens the drying time of the treatment. Drying in ambient conditions will only lengthen the drying time.

### Substrates

Various surfaces may be treated including for example metal, metal alloys, glass, plastics, rubber, porcelain, ceramic, tile, enamelled appliances, polyurethane, polyester, polyacrylic, melamine/phenolic resins, polycarbonate, painted surfaces, natural surfaces like wood, cellulose substrates, and the like.

1) The metal or metal alloy object or articles may be comprised of a metal or metal alloys selected from the group comprising: aluminium, magnesium, beryllium, iron, zinc, stainless steel, nickel, nickel-cobalt, chromium, titanium, tantalum, rare earth metal, silver, gold, platinum, tungsten, vanadium, copper, brass, bronze and the like or combinations or derivatives thereof or plated articles thereof

2) The plastic objects or articles may be comprised of polymers selected from the group comprising: transparent or non-transparent polyurethane, polycarbonate, polyethers, polyesters, polyvinyl chloride, polystyrene, polyethylene, polyvinyl acetate, silicone rubbers, rubber latex, polycarbonate, cellulose esters polycarbonate, polyester-polyether copolymers, ethylene methacrylates, polyolefins, and the like, silicone, natural and synthetic rubbers, nylon, polyamide or combinations thereof

3) The glass objects or articles may be comprised at least partially of: glass, such as optical glasses, optical lenses, polarizing glasses, mirrors, optical mirrors, prisms, quartz glass, ceramics and the like or combinations thereof.

The substrate may include an exterior surface or article member, such as for example: a window sash, structural member or windowpane of a building; an exterior member or coating of a vehicle such as automobile, railway vehicle, aircraft and watercraft; an exterior member, dust cover or coating of a machine, apparatus or article; and an exterior member or coating of a traffic sign, various display devices and advertisement towers, that are made, for example, of metal, plastics, glass, a combination thereof and other materials.

Examples of substrates, include, but are not limited to: medical devices, protection shields, window sheets, windowpane, greenhouse walls, freezer doors, food packaging foils and printing paper.

1) The metal objects can include for example: freezer doors, mirrors, condenser pipes, ship hulls, underwater vehicles, underwater projectiles, airplanes, wind turbine blades and the like.

2) The plastic objects can include for example: face shields, helmet shields, swim goggles, surgeon face shields, food packaging, plastic foil, greenhouse walls, greenhouse roofs, mirrors, wind shields, underwater moving objects, airplane windows, shields, and the like.

3) The glass objects can include for example: window glasses, greenhouse, glasses, glass sheets, face shields, optical glasses, optical, lenses, polarizing glasses, mirrors, optical mirrors, prisms, quartz glass, parabolic antennas, automobile head beam light glasses, automobile windshields, airplane control light glasses, solar panels, runway lights and the like.

The coating may also be applied on clear plastic or glass used for example as protective shields, windows, windshields, greenhouse panels, food packaging foils, goggles, optical glasses, contact lenses and the like.

Likewise the coating may be applied for example: on an exterior surface of a telescope lens, especially a riflescope, a spotting scope, or a binocular to reduce the likelihood of fogging or distortion due to the collection of moisture on the lens without significantly reducing light transmission through the lens in the visible range. That is, scopes used by sportsmen, the military and the like.

Exterior or interior parts of a building may also benefit form the coating for example: windowpanes, toilets, baths, wash basins, lighting fixtures, kitchenware, tableware, sinks, cooking ranges, kitchen hoods and ventilation fans, which are made from metal, glass, ceramics, plastics, a combination thereof, a laminate thereof or other materials.

### EXAMPLE 3

### “Easy-to Clean” and Dirt/Dust Repellent” Properties of a Surface Treatment According to the Present Invention

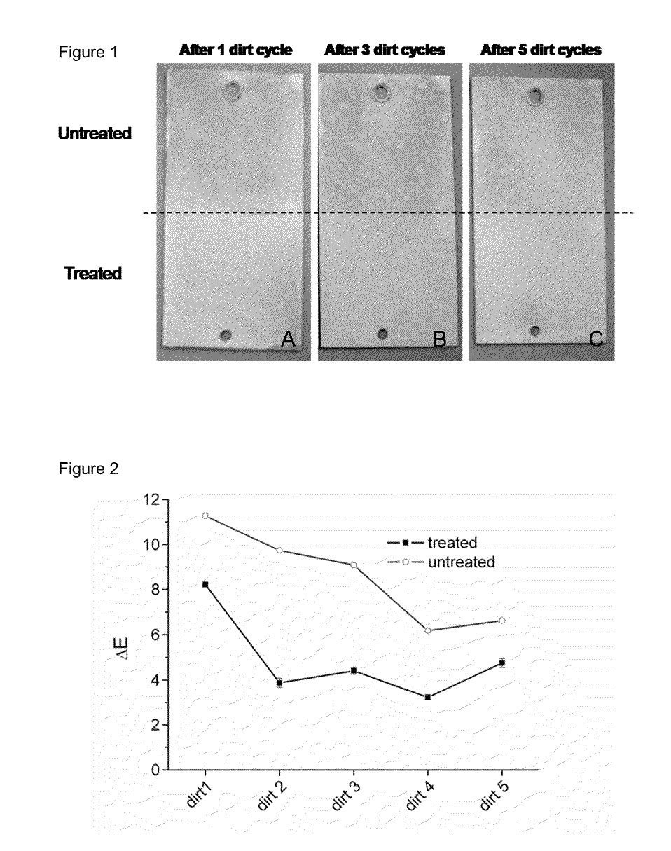

A surface treatment containing the polymer PBuMA₃₀-b-PMAA₁₁₉ was applied to one half of one side of a polyester powder coated aluminium panel. The panel was then placed in the bottom of a box containing a soiling material (for example garden soil with a mixture of several components such as clay, sand, formic acid, organic residues from plants). The panel was placed well beneath the soiling material. The box was attached to an electronic orbital shaker and was shaken for 30 seconds at a rate of 640 revolutions/minute. The panel was then removed from the box and tapped twice on a hard surface to remove excess soiling. Photographic images and visual observations were recorded (FIGS. 1a, 1b and 1c). The panel was then rinsed by spraying with 20 ml tap-water and allowed to dry. This process was considered as one dirt cycle and was repeated up to 5 times.

As seen in FIG. 1, the treated side (bottom of the picture) looks cleaner than the untreated side (top of the picture) of the panel. Indeed, it can be clearly seen that the dirt/dust that has been deposited and stacked on the untreated side remains whereas there is no deposit on the treated part of the panel.

Besides the visual observation and pictures seen in FIG. 1, colour measurements were recorded using a spectrophotometer after each dirt cycle to evaluate the colour difference ΔE between the surface prior to exposure to the dirt (that is, a clean surface) and the surface after exposure to dirt (that is the dirty surface).

The difference between the two surfaces is illustrated in FIG. 2 which shows a comparison of the total colour difference ΔE of the soiled panels. The colour difference is calculated from the CIE 1964 colour system. The system considers the lightness L*, the red-green value a* and the yellow-blue value, b*.

ΔE=(ΔL²+Δa²+Δb²)^{1/2} Equation 1

wherein

ΔL=L₁−L₂, Δa=a₁−a₂, Δb=b₁−b₂
And
ΔL, Δa, Δb are the colour differences in CIE L*a*b* colour space
L₁, a₁, b₁ are the L*a*b* values for sample 1 (clean panel before soiling)
L₂, a₂, b₂ are the L*a*b* values for sample 2 (panel after soiling or rinsing)

After soiling, the colour difference ΔE is less for the treated panels than for untreated panels. This corresponds to less initial soil adhesion on the treated panels, even after five dirt cycles. This demonstrates clearly that the surface treatment according to the present invention, once coated onto a surface provides “dirt repellent” properties compared with an uncoated surface.

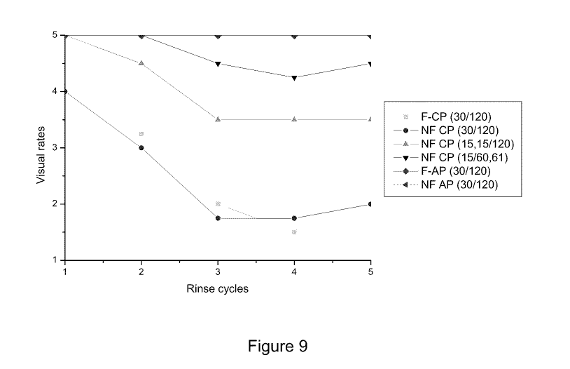

FIG. 9 illustrates the visual rating of performance or polymer coverage versus the number of water rinses to demonstrate the longevity of the “wetting effect” of the surface treatment applied polyester powder coated aluminium panels. FIG. 9 also illustrates the results for several polymers

From FIG. 9 it can be seen that the non-fluorinated (either anionic or cationic) (NF-AP or NF-CP) copolymers perform as well as the fluorinated copolymer derivative. That is, there is no loss in terms of performances using a copolymer which is more easily dispersed or solubilised in water (see Example 8 for the solubility comparison of the fluorinated and the non-fluorinated copolymers) and which address environmental/health concerns.

In addition, it can be seen that it is possible to ‘tune’ the longevity of the effect by ‘tuning’ the composition of the polymer which is utilised depending on the target application.
In FIG. 9, F CP (30/120) is PTFEMA₃₀-b-PDMAEMA₁₂₀
   NF CP (30/120) is PBuMA₃₀-PDMAEMA₁₂₀
   NF CP (15,15/120) is (P(BuMA₁₅-co-DEAEMA₁₅)-b-PDMAEMA₁₂₀
   NF CP (15/60,61) is PBuMA₁₅-b-P(TMACMA₆₀-co-PDMAEMA₆₁)
   F AP is PTFEMA₃₀-PMAA₁₂₀
   NF AP is PBuMA₃₀-PMAA₁₂₀

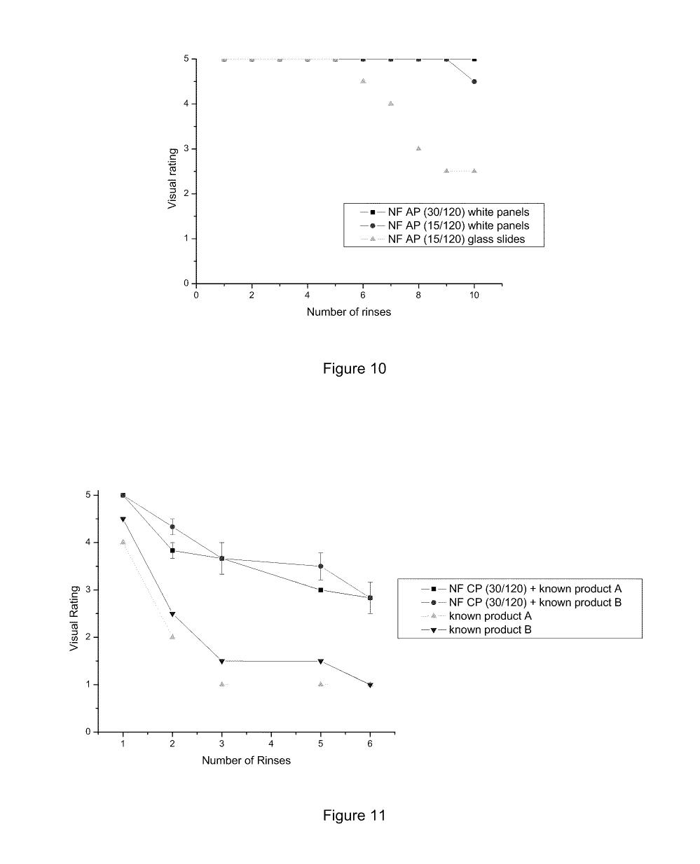

FIG. 10 illustrates the wettability performance of selected polymers on two substrates:
i) a polyester powder coated aluminium panel and
ii) a glass slide.

Both substrates were treated with a surface treatment containing the polymer BuMA₃₀-MAA₁₁₉. It can be clearly seen in FIG. 10 that depending on the substrate selected, and therefore depending on the target application, the longevity of the effect maybe be tuned.

FIG. 11 illustrates the performance of a surface treatment according to the present invention in comparison with commercially available products. Listed below are the compositions of known products A and B.

Product commercially available A has the following ingredients: Aqua, Propylene Glycol Butyl Ether, Alcohol Denat., Ethanolamine, Cocamidopropyl Hydroxysultaine, Parfum, Benzalkonium Chloride, Alkyl Dimethyl Ethylbenzyl, Ammonium Chloride, Tartrate, Sodium Chloride.

Product commercially available B has the following ingredients: Aqua, C9-11 pareth-3, sodium cumenesulfonate, sodium carbonate, parfum, sodium diethylenetriamine pentamethylene phosphonate, sodium palm kernelate, sodium dodecylbenzenesulfonate, sodium citrate, acrylic acid diquat copolymer, dipropylene glycol, lauramine oxide, benzisothiazolinone, butoxydiglycol, sodium hydroxide, sodium chloride, colorant, geraniol, limonene, linalool.

Polyester powder coated aluminium panels were then treated with:
1. Known product A only.
2. Known product B only.
3. A mixture of the known product A and the polymer PBuMA₃₀-b-PDMAEMA₁₂₀ at a concentration of 0.5 g/L
4. A mixture of the known product B and the polymer PBuMA₃₀-b-PDMAEMA₁₂₀ at a concentration of 0.5 g/L

All of the panels were then rinsed with sprayed tap water for 30 seconds. A visual rating was allocated. A rating of 5 means very good wettability, a rating of 1 means poor wettability.

It can be seen from FIG. 11 that the use of a polymer according to the present invention provides improved performance.

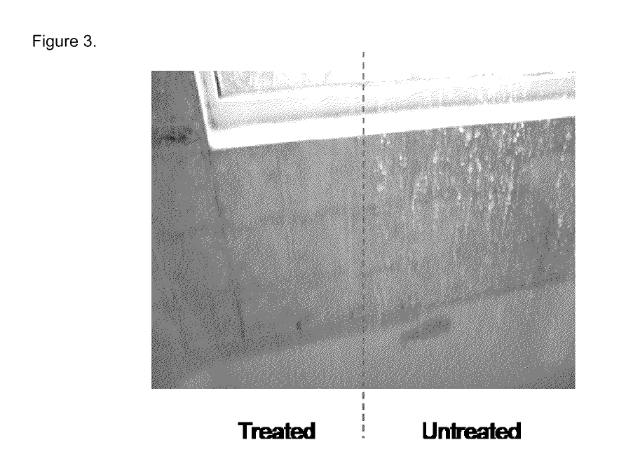

The surface treatment was also tested in a bathroom as seen in FIG. 3. In FIG. 3, a shower door has been half treated with a water-based treatment containing the polymer PBuMa₁₅-b-(TMACMA₆₀-co-DMAEMA₆₁).

After exposure to a shower in which shampoo and soap had been used, it was clearly observed that the treated side (left side on FIG. 3) was much cleaner that the untreated side (right side FIG. 3). Indeed, it can be observed that organic residues (from shampoo and soap) and insoluble mineral deposits (from hard water) have adhered and dried on the untreated side of the shower door whereas, no deposits or ‘build-up’ of material are present on the treated surface. Accordingly, this illustrates that the surface treatment reduces the appearance, formation, adhesion and build-up of insoluble mineral deposits, limescale, rust and soap scum when water is allowed to evaporate off most siliceous and non-siliceous surfaces.

Therefore, it is clear that the surface treatment according to the present invention provides superior “easy-to-clean” results.

### EXAMPLE 4

### “Water-Sheeting Behaviour” of the Surface Treatment According to the Present Invention

Panels were first cleaned in soap solution, rinsed with deionised water and then dried with a lint free tissue. The panels were half treated with a surface treatment comprising the polymer PBuMA₁₅-PMAA₁₁₉.

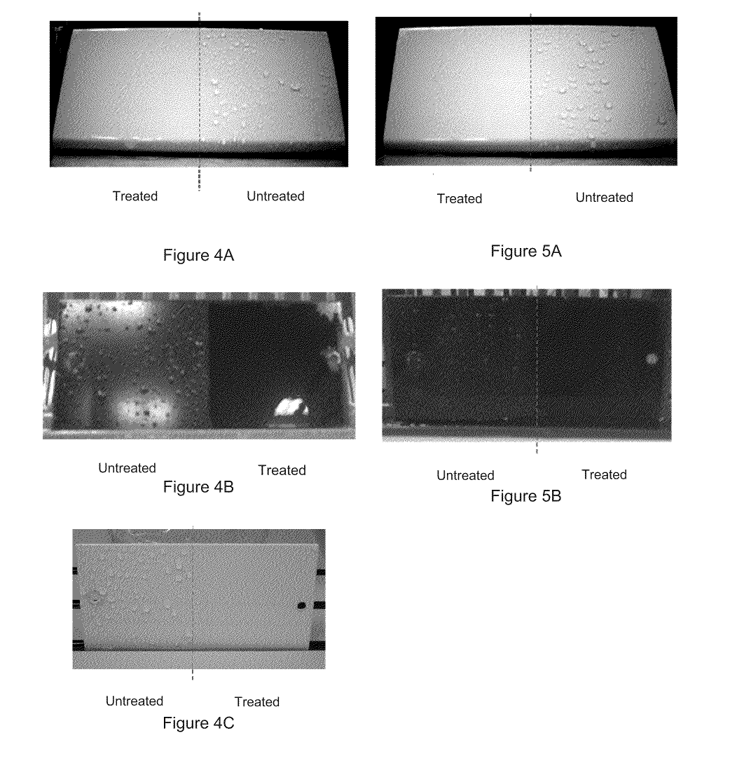

The treated substrates were exposed to continuous water spray for 30 seconds. When observed, the treated side was totally wetted by the water and demonstrated homogeneous coverage of the polymer with no degradation in performance, whatever the substrate, as seen on FIG. 4a (PVC substrate) and FIG. 4b (polyester powder coated aluminium panel).

Similar results were obtained with a treatment containing a cationic polymer PBuMA₁₅-b-P(TMACMA₆₀-co-PDMAEMA₆₁), as seen in FIG. 5a (PVC substrate) and FIG. 5b (painted aluminium panel).

Accordingly, it is also clear that the surface treatment according to the present invention provides a substrate with a good “water-sheeting” behaviour.

### EXAMPLE 5

### “Anti-Fog” Properties of Surface Treatment According to the Present Invention

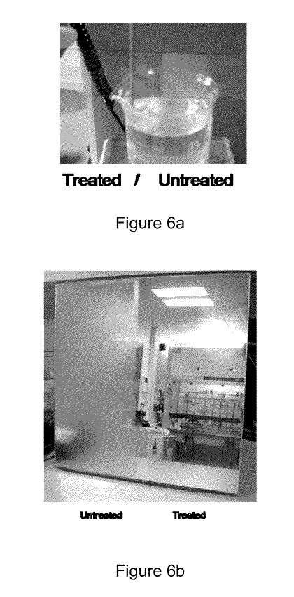

A surface treatment comprising the polymer BuMA₁₅-MAA₁₁₉ was applied on a polyester film which was used in food packaging (FIG. 6a) and on a glass mirror (FIG. 6b). The treated substrates were exposed to water vapour rising from a beaker containing tap water heated at 85° C. It was clearly observed that the treated side of the surface was totally fog free (that is, it was completely transparent) whereas on the untreated side of the surface, small droplets formed which produced poor visibility through the film.

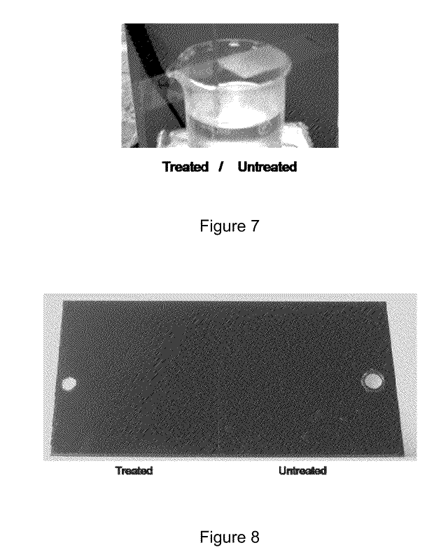

Similar results were obtained with a treatment comprising the cationic polymer PBuMA₁₅-b-P(TMACMA₆₀-co-DMAEMA₆₁), as seen in FIG. 7 (polyester film).

Accordingly, it was evident that the surface treatment according to the present invention provides “anti-fog” properties.

### EXAMPLE 6

### “Anti-Spotting” Properties of Surface Treatment According to the Present Invention

FIG. 8 illustrates the same panel as in FIG. 4b but after drying vertically at room temperature. On the untreated side (right), dried watermarks were observed leading to an unpleasant appearance or finish; whereas on the treated side (left) no drying-marks were present. This demonstrates the “spot-free finish effect” provided by using a surface treatment according to the present invention.

### EXAMPLE 7

### Investigates the “Antibacterial and Anti-Fungal” Properties of a Substrate Treated with the Block Copolymer of Example 1

A solution of PBuMA₁₅-b-P(TMACMA₆₀-co-DMAEMA₆₁) (NF CP 15/60,60) in ethanol was evaluated as an anti-bacterial and/or anti-fungal surface treatment in addition to its ability to promote water sheeting (FIG. 9).

The test was conducted as follows. Ceramic tiles (100×100 mm) were sterilised using alcohol in a Laminar flow cabinet. Two sterile tiles were then coated liberally with 2 ml of a solution of NF CP copolymer and allowed to air dry. The treated tiles and untreated control tiles were then assessed for the protection of the surface to microbial attack as detailed below.

One treated tile and two untreated tiles were surface inoculated with 1 ml of bacteria as detailed below in a Laminar flow cabinet. The inoculum was spread over the tile surfaces using sterile L shaped spreaders and allowed to air dry. One of the untreated tiles was swabbed to determine the number of colony forming units per tile by serial dilution and plate counting. The treated and untreated tiles were then washed by pouring 100 ml of sterile water over the tile surface and the number of surviving bacteria was determined by serial dilution and plate counting. The washed tile surface was then swabbed, which was serially diluted and plate counted to determine the colonies remaining on the tiles. This procedure was repeated using a fungi system as detailed below in Table 2.

TABLE 2

| | | INITIAL INOCULUM LEVEL |
|---|---|---|
| TEST SPECIES | | Colony forming units per tile |
| BACTERIA | | |
| Pseudomonas | NCIMB 10421 | 1.6 × 10⁷ |
| aeruginosa | | |
| Escherichia coli | NCIMB 8879 | |
| Staphylococcus | NCIMB 9518 | |
| aureus | | |
| Enterococcus hirae | NCIMB 8191 | |
| FUNGI | | |
| Candida albicans | NCPF 3179 | 4.9 × 10⁶ |
| Saccharomyces | CS Lab | |
| cerevisiae | Stock No. 4 | |
| Aspsergillus niger | NCPF 2275 | |
| Peniciiffium sp | CS Lab | |
| | Stock No. 25 | |

TABLE 3
Bacteria

| | COLONY FORMING UNITS REMOVED AFTER:- | | |
|---|---|---|---|
| SAMPLE | Wash | Swab | TOTAL |
| NF-CP | 7.3 × 10⁶ | 8.7 × 10³ | 7.3 × 10⁶ |
| CONTROL | 2.0 × 10⁷ | 3.3 × 10⁵ | 2.0 × 10⁷ |

TABLE 4
Fungi

| | COLONY FORMING UNITS REMOVED AFTER:- | | |
|---|---|---|---|
| SAMPLE | Wash | Swab | TOTAL |
| NF-CP | 3.5 × 10⁶ | 2.6 × 10⁴ | 3.5 × 10⁶ |
| CONTROL | 4.1 × 10⁶ | 3.9 × 10⁴ | 4.1 × 10⁶ |

TABLE 5

| | % REDUCTION AFTER WASH/SWAB : - | |
|---|---|---|
| SAMPLE | Bacteria | Fungi |
| NF-CP | 54.32 | 28.05 |
| CONTROL | No reduction | 15.53 |

The above results indicate:
The sample coated onto the surface of ceramic tiles resulted in lower numbers of colonies being recovered (surviving) after the wash and swab. The total reduction in the number of colonies was 54.32% for bacteria and 28.05% for fungi.
On the control ceramic tile showed there was no reduction in bacteria with a lower reduction of 15.53% for fungi.

Therefore the laboratory tests indicate that when a polymer coating is applied as a surface coating to ceramic tiles according to the present invention there seems to be an effect on the levels of bacteria and fungi inoculated onto the surface.

### EXAMPLE 8

### Analysis of the Concentration of Copolymers in a Water-Based Formulation Using the Non-Fluorinated Block Copolymer of Example 1 in Comparison to a Fluorinated Copolymer

The use of non-fluorinated copolymers facilitates the preparation of fully aqueous formulations in comparison to fluorinated copolymers. Indeed, whereas PTFEMA₃₀-b-PMAA₁₂₀ (F-AP 30/120) is not soluble in water, it has been possible to solubilise PBuMA₁₅-b-PMAA₁₂₀ (NF-AP 15/120) directly in water without using for example ethanol or surfactants.

Depending of the use of surface treatments, the concentration of copolymers required may vary. Using non-fluorinated copolymers can lead to a broader range of concentrations available in water based surface treatments.

The following table gives the maximum concentration (g/L) of copolymers F-AP and NF-AP in denaturated ethanol (IMS) and in water-based formulation. The water-based formulation was prepared by adding 5 mL of the solution of copolymers in ethanol at the maximum concentration into 45 mL water. Therefore, the water-based formulation contained 10 wt % alcohol. The maximum working concentration in a water based formulation can be eight times greater for the non-fluorinated polymer compared to the maximum working concentration for the fluorinated polymer.

TABLE 6

| | Maximum | Maximum concentration |
|---|---|---|
| Copolymers | concentration | in aqueous |
| type | in IMS (g/L) | formulation (g/L) |
| F-AP (30/120) | 75 | 7.5 |
| F-AP (15/120) | 600 | 60 |

### EXAMPLE 9

### Comparison of a NF-AP (15/120)-Based Surface Treatment Compared to Surface Treatments Prepared from Existing Additives which Promotes Stay-Clean Properties

Water-based surface treatments were prepared using NF-AP (15/20), F-AP (30/120) and as a comparison different commercially available existing additives and using two surfactants, Brij 30 and Glucopone CS 215, to facilitate the spreading of treatments on the tested substrates, in this experiment white-powder coated aluminium Q-panels. The concentration of each surfactant was 0.5 g/L. The existing additives were used at concentrations of 5 g/L, which is the recommended concentration advised by the suppliers. The copolymer of the present invention was used at 10-times lower concentration, 0.5 g/L in a water-based formulation containing 8 wt % ethanol. The table 7 summarises the content of each of the surface treatment formulations used in this comparison.

TABLE 7

| | Additive | | | | |
|---|---|---|---|---|---|
| | concentration | | Glucopone | | |
| Formulations | (g/L) | Brij 30 | CS 215 | Water | Ethanol |
| NF-AP (15/120) | 0.5 | ✓ | ✓ | ✓ | ✓ |
| F-AP (30/120) | 0.5 | ✓ | ✓ | ✓ | ✓ |
| Additive 1 | 5 | ✓ | ✓ | ✓ | — |
| Additive 2 | 5 | ✓ | ✓ | ✓ | — |
| Additive 3 | 5 | ✓ | ✓ | ✓ | — |

Each formulation was applied to a Q-panel by the flow-coating method of application and after drying, tap-water was sprayed onto the surfaces following the same procedure as described in example 4. A rating of 5 means very good wettability and water spreads readily over the panel, a rating of 1 means poor wettability and water beads on the panel.

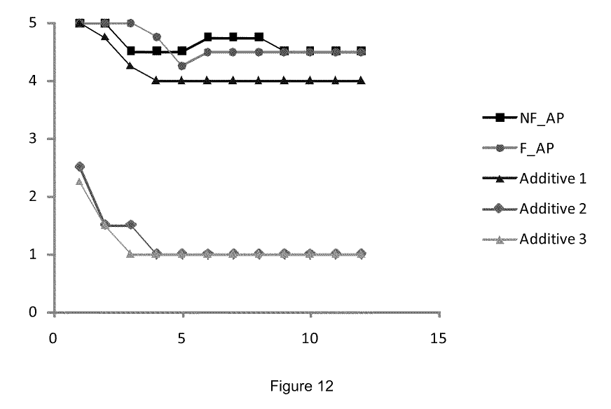

The results for 12 wetting cycles are shown in FIG. 12. FIG. 12 shows that the surface treatment comprising NF-AP (15/120) diblock copolymers performs as well as F-AP (30/130) copolymers in terms of water wetting properties and does not have any of the environmental constraints of F-AP (30/130).

Comparing the formulations prepared using the competitor additives, the surface treatment comprising additive 1 is the only formulation that achieves similar ratings to NF-AP(15/120). This however, is only achieved by using a much higher concentration of additive compared to the concentration of NF-AP (15/120) copolymers.

Formulations prepared with additives 2 and 3 show a lesser wetting effect than the surface treatments using NF-AP (15/120) and in particular, show a shorter longevity.

The water sheeting properties of the non-fluorinated product NF-AP performs as well as the fluorinated product F-AP and does not possess any of the environmental constraints associated with using fluorinated products. The only competitor product that displays similar performance to NF-AP is the formulation made with additive 1. The usage rate of this additive is, however, 10 times greater than NF-AP and therefore its use has a larger negative impact on the environment. This comparison demonstrates that there is an opportunity and requirement to improve stay-clean surface treatment products.

The present invention therefore provides novel uses of AB block copolymers which are able to self assemble into aggregate structures either in water, in a water/alcohol mixture or in a pure alcohol dispersion, for the preparation of a surface treatment which provides a functional coating, that includes the following properties and advantages and provides a surface coating or a surface treatment that imparts one or more functional effects:

(i) A coating that repels dust and dirt, that is, the coating reduces the adhesion and hence the build-up/depositition of soil/dust and dirt in order to provide a coated surface with a cleaner appearance and also a coating that is easier to clean.

(ii) A coating that has improved water-sheeting behaviour; meaning that water does not de-wet or experience beading on the surface, rather the water forms a continuous sheet meaning that the surface is wetted by water easily; in other words provides good water wettability.

(iii) A coating that once applied to a surface prevents the build-up of crystalline scale deposits and the associated surface fouling effects that are visible with the build-up of lime scale.

The surface treatment is able to reduce the appearance and prevent the build-up and deposition of lime scale. The formation of crystalline deposits due to chemicals present in water which build up over a period of time, particularly in bathrooms, toilets, sinks and particularly in places where there are flows of domestic tap water, are also reduced.

(iv) A coating that is able to provide ‘anti-fog’ properties. A haze, mist or fog is defined as the formation of small droplets of water on a transparent surface in the presence of water vapour that results in the transparency of the surface being reduced. Consequently, the terms “anti-hazing”, “anti-mist”, “anti-fogging”, “fog resistance” or “fog up free” properties refer to the properties of a transparent surface which has been treated in such a way so that in the presence of water vapour either:

(1) beads of water are not able to form on the surface and instead water vapour on contact with the surface forms a thin continuous sheet in the form of a film at the surface which is transparent; or

(2) water is repelled on contact with the surface which results in beads which have the ability to readily roll off the surface. In both cases, the haze which is caused by the formation of small droplets of water on a surface in the presence of water vapour is reduced.

More specifically, the use of a hydrophilic surface treatment according to the present invention means that when the coating is applied to a surface for example, but not limited to: metal, glass or plastic surfaces, the surface coating or a surface treatment prevents water droplet formation on the selected surfaces when the surfaces are exposed to conditions which can lead to the ‘fogging’ of the surface. Such conditions include for example: the exposure of the surface to air of high humidity, the exposure of the surface to water vapour or the transfer of the surface from a low temperature environment to a higher temperature environment causing the surface to ‘fog up’, that is the surface becomes clouded by condensation formed from the cooling of water alighting on the surface.

The applied hydrophilic surface treatment of the present invention is useful for preventing water condensation or fogging on metallic, plastic, and glass surfaces and the like. That is, the applied hydrophilic surface treatment of the present invention does not prevent water condensation, but water that has condensed forms a continuous sheet rather then beads. As an aside, when the selected surface coating or surface treatment is clear plastic or glass, the treatment applied to the surface ensures that these particular surfaces maintain a good transparency.

(v) In addition it has been found that the combination of the durable hydrophilic effect and the fast and uniform drying effect of the polymeric surface coating or surface treatment of the present invention provides treated substrates with ‘anti-spotting’ properties and therefore provides treated surfaces with an aesthetically pleasing finish, that is a treated surface has a ‘spot-free’ finish, more specifically a treated surface which does not display the appearance of water-marks, even after the treated surface has been contacted at a later point in time with water.

A further feature of the presently claimed surface coating or surface treatment is that the treatment may be applied to a wide range of substrate surfaces, for example but not limited to: plastic, metal or other materials.

It has been found that the hydrophilic surface treatment of the present invention adheres ‘strongly’ to for example surfaces that include: metals, metal alloys, glass, plastics, rubber, porcelain, ceramic, tile, enamelled appliances, polyurethanes, polyesters, polyacrylics, melamine/phenolic resins, polycarbonates, painted surfaces, and wood.

Consequently, the hydrophilic surface treatment of the present invention finds particular use in a wide range of application areas such as for example; building and DIY treatments, the car industry for the treatment of interior and exterior metal and glass, bathroom and wet room applications, and general household surface cleaning products.

The treatment also finds use in other application areas such as for example but not limited to: the food packaging and foils industry, and as protective shields.

Fogging is a phenomenon observed commonly in applications of plastic films in the food packaging and agricultural sectors. The term ‘Fog’ used herein describes the condensation of water vapour, in the form of small discrete water droplets, creating a translucent appearance, on a plastic film surface when an enclosed mass of air cools to a temperature below its dew point. The extent of the phenomenon depends on the actual temperature and relative humidity of the enclosed air mass, as well as the temperature of the plastic film. Examples of the problems caused by this phenomenon include: fogging of food packaging in chiller cabinets and condensation within greenhouse complexes.

Food packaging needs to present its contents hygienically and aesthetically. Fogging reduces a consumer's ability to see the product and will give an impression of lower quality. In some applications condensation of water within the packaging may lead to actual reduction in quality.

In the agricultural sector, the undesirable effects of fogging include for example; reduced total light transmission in greenhouses and water dripping that can lead to plant damage. Further plant damage may be induced by the focussing effect of the water droplets, rather like an array of lenses concentrating solar energy on foliage. The end result of all these effects for food producers is a lower potential yield and reduced product quality.

Eye protection is also increasingly important in today's industrial environment. Best practices dictate that employees exposed to eye hazards such as airborne debris, fumes, or even excess moisture, must wear goggles, shields or safety glasses. Unfortunately, these eye safety systems are often plagued with fogging. That fogging often causes the frustrated employee to stop wearing their eye protection thus exposing themselves to potential injury and the company to a lawsuit for workers compensation claims. When goggles, shields or safety glasses do not provide adequate fog and condensation protection, employees may spend valuable production time constantly clearing the fog from these lenses. Protective eyewear is often available with glass, plastic and polycarbonate lenses. However none of these provide adequate fog protection. The use of the “anti-fog” surface treatment such as described in the present invention will be suitable on polycarbonate lenses and other plastic lenses like those found in high-end safety eyewear and will aid in keeping protective eyewear fog free and a visibility clear.

While exemplary embodiments incorporating the principles of the present invention have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

### REFERENCES

1. Emma Dorey, Chemistry & industry, issue 18, 5 (September 2006);
2. Ralf Blossey, Nature Materials, vol 2, 301-306, (2003);
3. Jon Evans, Chemistry & industry, issue 18, 16-17 (September 2006);
4. Mathilde Callies, et al., Soft matter, vol 1, 55-61, (2005);
5. Taolei Sun, et al., Chem. Comm., 1723-1725, (2005);
6. a/ S. Herminghaus, Europhys. Lett., 52, 165, (2000); b/ J. Bico, et al., Colloids Surf., A, 206, 41, (2002); c/ H. Li, et al., Angew. Chem. Int. Ed., 40, 1743, (2001); d/ L. Feng, et al., Angew. Chem. Int. Ed., 41, 1221, (2002); e/ L. Feng, et al., Angew. Chem. Int. Ed., 42, 800, (2003); f/ T. Onda, et al., Langmuir, 12, 2125, (1996);
7. Igor Luzinov, et al., Prog. Polym. Sci., vol 29, 635-698, (2004);
8. a/ Anthony M. Granville, et al., Macromol. Rapid. Comm., vol 25, 1298-1302, (2004); b/ Lei Thai, et al., NanoLetters, vol 4, 7, 1349-1353, (2004); c/ Motoshi Yamanaka, et al., Chem Comm, 2248-2250, (2006);
9. Akira Nakajima, et al., Langmuir, 16 (17), 7044-7047, (2000);
10. a/ Eiji Hosono, et al.; JACS, vol 127, 13458-13459, (2005); b/ Xi Yu, et al., Adv. Mater. Vol 17, 1289-1293, (2005); c/ A. A. Abramzon, Khimia i Zhizu (1982), no. 11, 38 40;
11. J. Rodriguez-Hernandez, et al., Prog. Polym. Sci., 30, 691-724, (2005);

What is claimed is:

1. A surface coating composition comprising an AB block copolymer and a liquid medium, wherein the AB block copolymer comprises:
(a) a substantially hydrophobic block A, and
(b) a substantially hydrophilic block B;
wherein the hydrophobic block A comprises one or more monomer of formula A 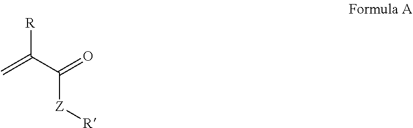
wherein R is H or C₁ to C₄ alkyl; Z is O, P or N; and
R′ is selected from the group comprising: C₁ to C₁₈ linear or non linear alkyl; C₁ to C₁₈ alkylamino alkyl; C₁ to C₁₈ alkoxyalkyl; C₁ to C₁₈ dihydroxyalkyl; C₁ to C₁₈ sillylalkyl; epoxy alkyl, phosphoryl or phosphoryl alkyl; a styrene based monomer; and a vinyl phosphonate or phosphoric acid monomer;
wherein the liquid medium comprises either:
(i) water;
(ii) an organic solvent;
(iii) an organic solvent substantially free from water; or
(iv) an organic solvent and water; and
wherein the liquid medium further optionally comprises one or more additive, surfactant or wetting agent.

2. The surface coating composition of claim 1 wherein the hydrophilic block B comprises one or more monomers of Formula B 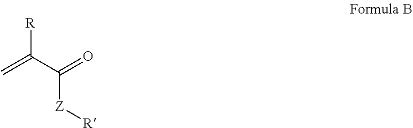
wherein R is H or C₁ to C₄ alkyl;
Z is O, N or P; and
R′ is selected from the group comprising H and a C₁ to C₁₇ alkyl group with a pendent phosphoryl group, hydroxy group, silyl group, epoxy group or amine group.

3. The surface coating composition of claim 1 wherein when the hydrophobic block A comprises an alkylacrylic or acrylate monomer of Formula 1 wherein R is H or C₁ to C₄ alkyl, and 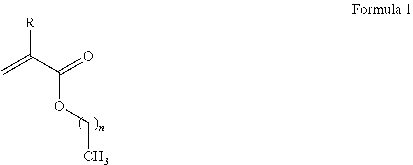
wherein n is 1 to 11 or 1 to 5.

4. The surface coating composition of claim 1 wherein when the hydrophobic block A comprises a monomer of Formula 2 wherein R is H or C₁ to C₄ alkyl, 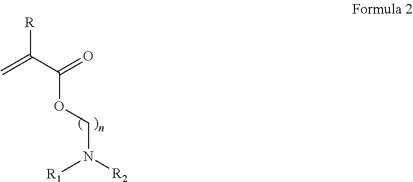
and wherein R₁ and R₂ are each independently: H; C₁ to C₆ alkyl group; phenyl; benzyl or cyclohexyl and n is 1 to 17 or R₁ and R₂ are each independently a C₁ alkyl group and n is 1 to 5.

5. The surface coating composition of claim 1 wherein when the hydrophobic block A comprises a monomer of Formula 3a or 3b wherein R is H or C₁ to C₄ alkyl, n is 1 to 18 or 1 to 4 and x and y are each independently 0 to 16 or 0 to 6.

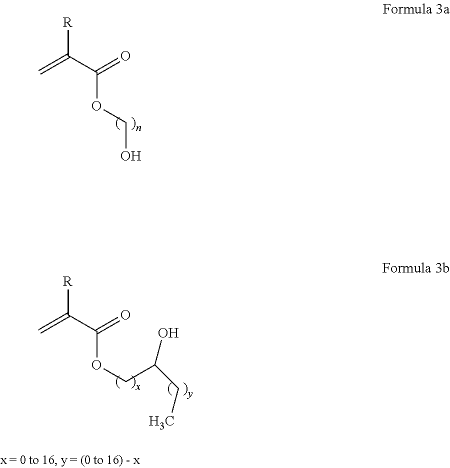

6. The surface coating composition of claim 1 wherein when the hydrophobic block A comprises an alkylacrylic or acrylate monomer of Formula 4a or 4b wherein R is H or C₁ to C₄ alkyl, x and y is each independently 0 to 17 or 0 to 7 in Formula 4a or 0 to 16 or 0 to 6 in Formula 4a and Formula 4b.

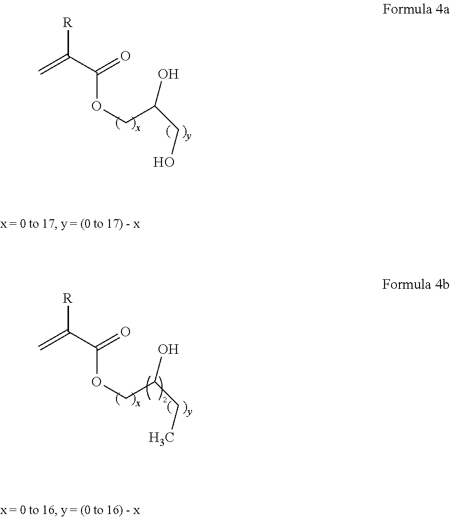

7. The surface coating composition of claim 1 wherein when the hydrophobic block A comprises an alkylacrylic or acrylate monomer for Formula 5a or 5b respectively, and R is H or C₁ to C₄ alkyl, R₁ is H or C₁ to C₄ alkyl and x and y are each independently 0 to 16 or 1 to 6.

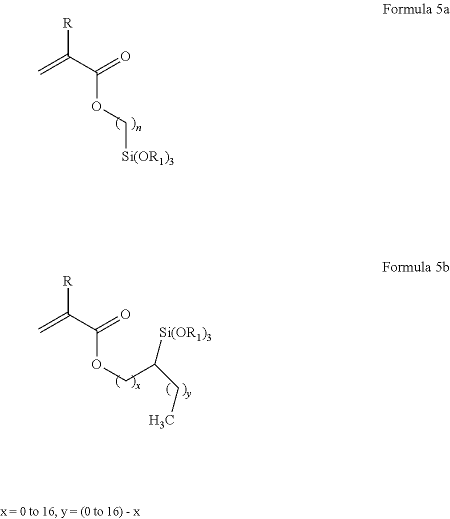

8. The surface coating composition of claim 1 wherein when the hydrophobic block A comprises an alkylacrylic or acrylate monomer of Formula 6a or 6b respectively, and R is H or C₁ to C₄ alkyl, x and y are each independently 0 to 16, more preferably 0 to 6.

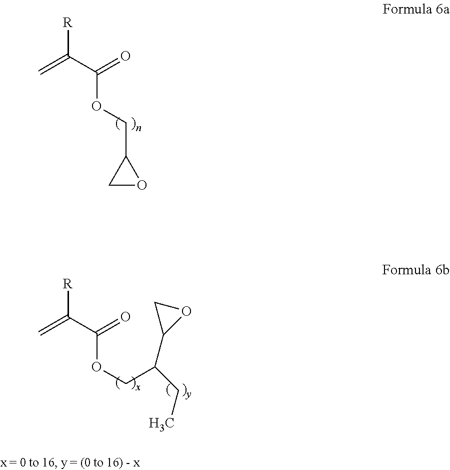

9. The surface coating composition of claim 1 wherein when the hydrophobic block A comprises a monomer of Formula 7a and 7b having a phosphoryl or phosphoryl alkyl group, respectively wherein R is H or C₁ to C₄ alkyl group, and R₁ is H or C₁ to C₆ alkyl.

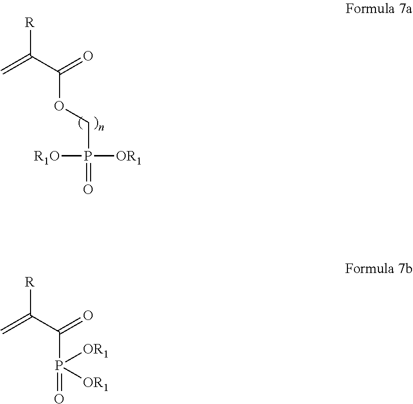

10. The surface coating composition of claim 2 wherein when the hydrophilic block B comprises Formula 8 having an alkylacrylic acid or acrylic acid wherein R is H or C₁ to C₄ alkyl as shown in Formula 8

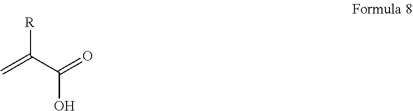

11. The surface coating composition of claim 2 wherein when the hydrophilic block B comprises a monomer of Formula 9a, 9b, 9c or 9d comprising a C₁ to C₁₇ alkyl group with a pendent amine or amide group wherein R is H or C₁ to C₄ alkyl; n is 0 to 17, and R₁, R₂, R₃ and R₄ are each independently H; linear or non-linear C₁ to C₆ alkyl group, phenyl, benzyl or cyclohexyl, and X⁻ is chosen from the group selected from Cl, Br, I, ½SO₄, HSO₄ and CH₃SO₃, sulfonate, sulphate, carboxylate (acetate, glycolate), hydroxide, or phosphate.

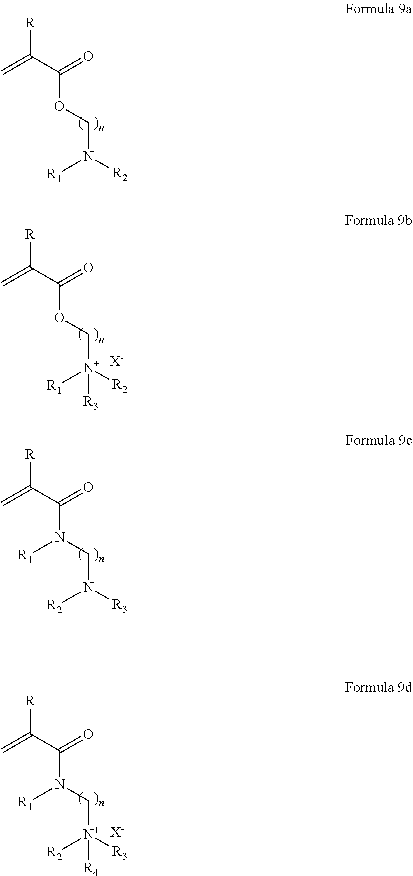

12. The surface coating composition claim 2 wherein when the hydrophilic block B comprises a monomer of Formula 10a or 10b having a C₁ to C₁₇ alkyl group with a pendent phosphoryl or phosphoryl alkyl wherein R is H or C₁ to C₄ alkyl and R₁ is H or C₁ to C₆ alkyl.

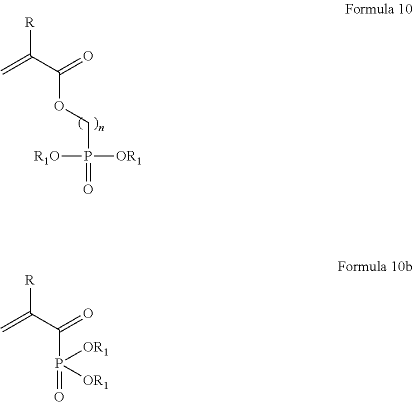

13. The surface coating composition of claim 2 wherein when the hydrophilic block B comprises a monomer of Formula 11a, 11b, 11c or 11d having a C₁ to C₁₇ alkyl group with a pendent hydroxyl group wherein R is H or C₁ to C₄ alkyl and n is 1 to 16.

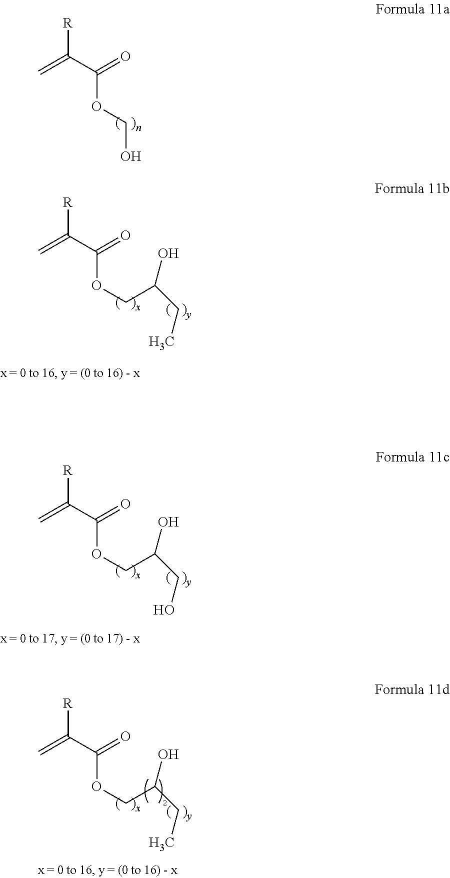

14. The surface coating solution of claim 2 wherein when the hydrophilic block B comprises a monomer of Formula 12a or 12 b having a C₁ to C₁₇ alkyl group with a pendent silyl group wherein R is H or C₁ to C₄ alkyl, x and y are each independently 0 to 16 and n is 1 to 6.

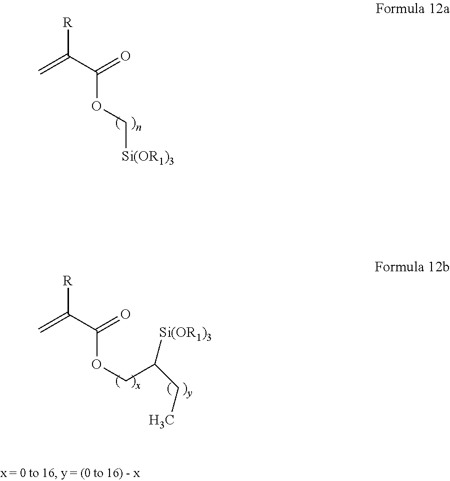

15. The surface coating solution of claim 2 wherein when the hydrophilic block B comprises an alkylacrylate or acrylate of Formula 13a or 13b having a C₁ to C₁₇ alkyl group with a pendent epoxy group wherein R is H or C₁ to C₄ alkyl, x and y are each independently 0 to 16 and n is 1 to 17.

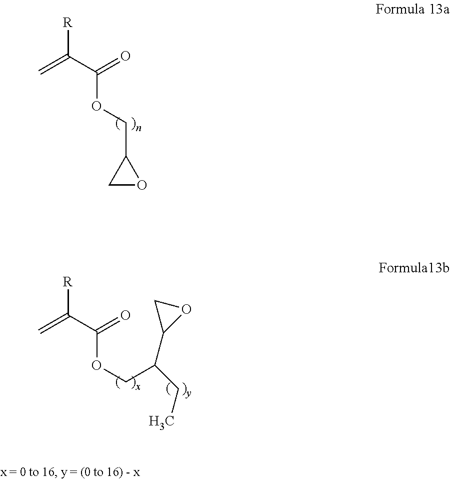

16. The surface coating solution of claim 1 wherein the polymers comprising the AB block copolymer are comprised of monomers, and wherein the ratio of the monomers comprising each polymer of the block copolymer AB is such that a volume fraction of the hydrophobic block A and the hydrophilic block B leads to the formation of organised aggregates.

17. The surface coating solution of claim 16 wherein the volume fraction of the hydrophobic block A and the hydrophilic block B leads to the formation of micelles.

18. The surface coating solution of claim 1 wherein a ratio of the monomers comprising the block copolymer AB comprises 5 to 100 monomers of Formula A and 15 to 300 monomers of Formula B.

19. The surface coating solution of claim 18 wherein the ratio of the monomers comprising the block copolymer AB comprises 15 to 50 monomers of Formula A and 80 to 200 monomers Formula B.

20. The surface coating solution of claim 18 wherein the ratio of the monomers comprising the block copolymer AB comprises 15 to 30 monomers of formula A and 100 to 120 monomers of formula B.

21. The surface coating solution of claim 1 wherein hydrophobic block A comprises one or more monomers selected from the group comprising alkyl alkylacrylate, alkylaminoalkyl alkylacrylate and a silylacrylate.

22. The surface coating solution of claim 1 wherein substantially hydrophobic block A comprises an alkyl alkylacrylate selected from the group comprising a methacrylate; butyl methacrylate (BuMA) and octadecylmethacrylate (ODA).

23. The surface coating solution of claim 22 wherein hydrophobic block A preferably comprises one or more alkylaminoalkylacrylate alkylacrylate monomer according to Formula A wherein the alkylaminoalkylacrylate alkylacrylate monomer comprises an alkylaminoalkyl methacrylate or a diethylaminoethylmethacrylate (DEAEMA).

24. The surface coating solution of claim 1 wherein when hydrophobic block A comprises a silyl (alkyl)acrylate monomer, the silyl (alkyl)acrylate monomer comprising a trialkoxysilyl group, a trimethoxysilyl group, a (trimethoxysilyl) propyl methacrylate (TMSPMA) or a (trimethoxysilyl) propyl acrylate (TMSPA).

25. The surface coating solution of claim 1 wherein when hydrophobic block A comprises a styrenic derivative, the styrenic derivative comprising styrene, methyl styrene, or styrenic derivatives substituted in ortho, meta, para or a combinantion of ortho, para or meta positions.

26. The surface coating solution of claim 1 wherein hydrophobic block A is preferably comprised of:
a homopolymer of an acrylate derivative;
a homopolymer of styrenic derivatives; and
a random, alternating, gradient or block copolymer of A1A2, A1A3, A1A4, A2A4 or A3A4 wherein A1 is an alkyl acrylate; A2 an alkylamino acrylate; A3 a silyl acrylate; and A4 a styrenic derivative.

27. The surface coating solution of claim 1 wherein block B is preferably comprised of one or more polymers selected from the group comprising of:
hydrophilic organic monomers, oligomers, prepolymers or copolymers derived from vinyl alcohol, N-vinylpyrrolidone, N-vinyl lactam, acrylamide, amide, styrenesulfonic acid, combinations of vinylbutyral and N-vinylpyrrolidone, methacrylic acid, acrylic acid, vinylmethyl ether, vinylpyridylium halide, melamine, maleic anhydride/methyl vinyl ether, vinylpyridine, ethyleneoxide, ethyleneoxide ethylene imine, glycol, vinyl acetate, vinyl acetate/crotonic acid, methyl cellulose, ethyl cellulose, carboxymethyl cellulose, hydroxyethyl cellulose, hydroxypropyl cellulose, hydroxymethyl ethyl cellulose, hydroxypropylmethyl cellulose, cellulose acetate, cellulose nitrate, hydroxyalkyl(alkyl)acrylate such as hydroxyethyl(meth)acrylate, hydroxypropyl(meth)acrylate, alkylaminoalkyl(alkyl)acrylate, 2-(dimethyl amino)ethyl methacrylate, 2-(diethyl amino)ethyl methacrylate, 2-(diisopropyl amino)ethyl methacrylate, 2-(N-morpholino)ethyl methacrylate, or a derivative thereof, ethylene glycol(meth)acrylates (for example triethylene glycol(meth)acrylate) and (meth)acrylamide), N-alkyl(meth)acrylamides (for example N-methyl(meth)acrylamide and N-hexyl(meth)acrylamide), N,N-dialkyl(meth)acrylamides (for example N,N-dimethyl(meth)acrylamide and poly-N,N-dipropyl(meth)acrylamide), N-hydroxyalkyl(meth)acrylamide polymers, such as poly-N-methylol(meth)acrylamide and poly-N-hydroxy ethyl(meth)acrylamide, and N,N-dihydroxyalkyl(meth)acrylamide polymers, such as poly-N,N-dihydroxyethyl(meth)acrylamide, ether polyols, polyethylene oxide, polypropylene oxide, and poly(vinyl ether), alkylvinyl sulfones, alkylvinylsulfone-acrylates, (alkyl)acrylate with a pendent phosphorus group such as vinylphosphonate, vinylphosphonic acid, vinylphosphine oxide or any (alkyl)acrylate with a ester function —COOR such as R is CₓH₂ₓPO₃R₂ wherein x is 2 to 10, most preferably x is 2, and R is a hydrogen or an alkyl group having 1 to 4 carbon atoms, preferably methyl; and related compounds or a combination thereof.

28. The surface coating solution of claim 27 wherein the hydrophilic block B is comprised from monomers selected from the group consisting of: hydrophilic organic monomers, oligomers, prepolymers or copolymers derived from vinyl alcohol, N-vinylpyrrolidone, N-vinyl lactam, acrylamide, styrenesulfonic acid, combinations of vinylbutyral and N-vinylpyrrolidone, methacrylic acid, acrylic acid, vinylmethyl ether, vinylpyridylium halide, vinylpyridine, vinyl acetate, vinyl acetate/crotonic acid, hydroxyalkyl(alkyl)acrylate such as hydroxyethyl(meth)acrylate, hydroxypropyl(meth)acrylate, alkylaminoalkyl(alkyl)acrylate, 2-(dimethyl amino)ethyl methacrylate, 2-(diethyl amino)ethyl methacrylate, 2-(diisopropyl amino)ethyl methacrylate, 2-(N-morpholino)ethyl methacrylate, or a derivative thereof, ethylene glycol(meth)acrylates (for example triethylene glycol(meth)acrylate) and (meth)acrylamide), N-alkyl(meth)acrylamides (for example N-methyl(meth)acrylamide and N-hexyl(meth)acrylamide), N,N-dialkyl(meth)acrylamides (for example N,N-dimethyl(meth)acrylamide and poly-N,N-dipropyl(meth)acrylamide), N-hydroxyalkyl(meth)acrylamide polymers, such as poly-N-methylol(meth)acrylamide and poly-N-hydroxy ethyl(meth)acrylamide, and N,N-dihydroxyalkyl(meth)acrylamide polymers, such as poly-N,N-dihydroxyethyl(meth)acrylamide, ether polyols, polyethylene oxide, polypropylene oxide, and poly(vinyl ether), alkylvinyl sulfones, alkylvinylsulfone-acrylates, (alkyl)acrylate with a pendent phosphorus group such as vinylphosphonate, vinylphosphonic acid, vinylphosphine oxide or any (alkyl)acrylate with a ester function —COOR such as R is CₓH₂ₓPO₃R₂ wherein x is 2 to 10, most preferably x is 2, and R is a hydrogen or an alkyl group having 1 to 4 carbon atoms, preferably methyl.

29. The surface coating solution of claim 28 wherein the hydrophilic block B is comprised of monomers selected from the group consisting of: hydrophilic organic monomers, oligomers, prepolymers or copolymers derived from acrylamide, methacrylic acid, acrylic acid, hydroxyalkyl(alkyl)acrylate such as hydroxyethyl(meth)acrylate, hydroxypropyl(meth)acrylate, alkylaminoalkyl(alkyl)acrylate, 2-(dimethyl amino)ethyl methacrylate, 2-(diethyl amino)ethyl methacrylate, 2-(diisopropyl amino)ethyl methacrylate, 2-(N-morpholino)ethyl methacrylate, or a derivative thereof, N-alkyl(meth)acrylamides (for example N-methyl(meth)acrylamide and N-hexyl(meth)acrylamide), N,N-dialkyl(meth)acrylamides (for example N,N-dimethyl(meth)acrylamide and poly-N,N-dipropyl(meth)acrylamide), N-hydroxyalkyl(meth)acrylamide polymers, such as poly-N-methylol(meth)acrylamide and poly-N-hydroxy ethyl(meth)acrylamide, alkylvinyl sulfones, alkylvinylsulfone-acrylates and (alkyl)acrylate with a pendent phosphorus group such as vinylphosphonate, vinylphosphonic acid, vinylphosphine oxide or any (alkyl)acrylate with a ester function —COOR such as R is CₓH₂ₓPO₃R₂wherein x is 2 to 10, most preferably x is 2, and R is a hydrogen or an alkyl group having 1 to 4 carbon atoms, preferably methyl.

30. The surface coating solution of claim 28 wherein the hydrophilic block B is comprised of monomers selected from hydrophilic organic monomers, oligomers, prepolymers or copolymers derived from methacrylic acid, acrylic acid, alkylaminoalkyl(alkyl)acrylate, 2-(dimethyl amino)ethyl methacrylate, 2-(diethyl amino)ethyl methacrylate, 2-(diisopropyl amino)ethyl methacrylate, 2-(N-morpholino)ethyl methacrylate, or a derivative thereof.

31. The surface coating solution of claim 1 wherein the AB block copolymer is in the form of a linear block copolymer (diblock, triblock or multiblock), a miktoarm copolymer (star copolymer), a ladder (H-shaped) copolymer or a graft and comb (co)polymer.

32. The surface coating solution of claim 1 wherein the distribution of component monomers within each copolymer block is in the form of homo, random, gradient, alternative, block, graft and comb (co)polymers.

33. The surface coating solution of claim 31 wherein the block copolymer is preferably selected from the group comprising: AB blocks, ABA blocks, ABC blocks copolymers.

34. The surface coating solution of claim 1 wherein the block copolymer comprises at least one block that absorbs to a target surface.

35. The surface coating solution of claim 1 wherein the composition further comprises a primer.

36. The surface coating solution of claim 1 wherein the composition forms micelles.

37. The surface coating solution of claim 1 comprising aggregate structures of diameter between 3 and 300 nm.

38. The surface coating solution of claim 1 wherein the polymers are prepared by controlled living radical polymerisation reactions.

39. The surface coating solution of claim 1 wherein when the liquid medium comprises water, water and organic solvent, an organic solvent, or an organic solvent free from water, and wherein the block copolymer is preferably completely dissolved in the liquid medium.

40. The surface coating solution of claim 1 wherein the organic solvent preferably comprises water-miscible organic solvents selected from the group comprising: C₁₋₆ alcohol, preferably, methanol, ethanol, n-propanol, iso-propanol, n-butanol, and sec-butanol; alkylketones, arylalkylketones, ketoalcohols, cyclic ketones, heterocyclic ketones, ethers, cyclic ethers, esters, and the like and combinations thereof.

41. The surface coating solution of claim 1 wherein the liquid medium preferably comprises:
water or a mixture of water/alcohol or pure alcohol wherein the alcohol is preferably selected from the group comprising: methanol, ethanol, industrial methylated spirit, propanol, isopropanol, tertbutanol, ethylene glycol and glycol ethers.

42. The surface coating solution of claim 1 wherein the relative proportions of block copolymers AB as components (a) and of liquid medium as component (b) in the composition comprises between from 1:100,000 to 1:1, from 1:10,000 to 1:2, or from 1:5,000 to 2:10.

43. The surface coating solution of claim 1 wherein the relative proportions of component (a) and component (b) in the composition comprises from 1:5,000 to :1:10.

44. The surface coating solution of claim 1 wherein the composition further comprises additional components selected from dispersants, perfumes surfactants and stabilisers.

45. A method of coating a surface of a substrate with the surface coating solution of claim 1 wherein the method comprises dipping, spraying, wiping, spin coating, roller coating, curtain flowing and brush coating.

46. The method of claim 45 wherein the substrate is selected from the group comprising: glass, plastics, metals, ceramics, concrete, paper, wood, minerals and combinations thereof.

47. The method of claim 45 wherein the substrate is coated or painted prior to application of the composition with a primer.

48. A coating formed on a substrate, the coating comprising an AB block copolymer, wherein the AB block copolymer comprises:
(a) a substantially hydrophobic block A, and
(b) a substantially hydrophilic block B;
wherein the hydrophobic block A comprises one or more monomer of formula A 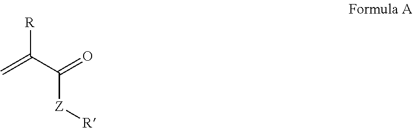
wherein R is H or C₁ to C₄ alkyl; Z is O, P or N; and
R′ is selected from the group comprising: C₁ to C₁₈ linear or non linear alkyl; C₁ to C₁₈ alkylamino alkyl; C₁ to C₁₈ alkoxyalkyl; C₁ to C₁₈ dihydroxyalkyl; C₁ to C₁₈ sillylalkyl; epoxy alkyl, phosphoryl or phosphoryl alkyl; a styrene based monomer; and a vinyl phosphonate or phosphoric acid monomer; and
wherein the hydrophilic block B comprises one or more monomers of Formula B 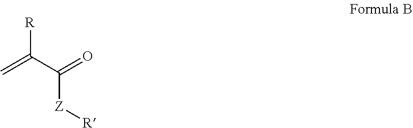
wherein R is H or C₁ to C₄ alkyl;
Z is O, N or P; and
R′ is selected from the group comprising H and a C₁ to C₁₇ alkyl group with a pendent phosphoryl group, hydroxy group, silyl group, epoxy group or amine group.
